(19) United States
(12) Patent Application Publication
(10) Pub. No.: US 2010/0098689 A1
(43) Pub. Date: Apr. 22, 2010

(54) 4-1 bb ligand in inflammatory diseases

(75) Inventors: Young Jun Kang, San Diego, CA (US); Jiahuai Han, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 12/311,170

(22) PCT Filed: Oct. 16, 2007
(86) PCT No.: PCT/US2007/022103

§ 371 Date: Sep. 29, 2009

Related U.S. Application Data
(60) Provisional application No. 60/917,561, filed on May 11, 2007.
(60) Provisional application No. 60/852,022, filed on Oct. 16, 2006.

(51) Int. Cl. A61K 39/395; A61K 31/7052; A61K 38/14; C07K 16/00; A61P 29/00

(52) U.S. Cl. 424/133.1; 424/173.1; 514/44 R; 514/8; 424/158.1; 530/387.3; 530/395

(57) ABSTRACT

The invention provides 4-IBBL blocking agents, as well as pharmaceutical compositions and articles of manufacture comprising such blocking agents as new therapeutic interventions for sustained inflammation. Thus, the invention also provides methods for reducing sustained production of tumor necrosis factor.

### RELATED APPLICATIONS

This application claims priority to U.S. Provisional Application Ser. No. 60/852,022, filed Oct. 16, 2006, and U.S. Provisional Application Ser. No. 60/917,561, filed May 11, 2007, both of which are incorporated herein by reference in their entirety.

### STATEMENT REGARDING FEDERALLY SPONSORED RESEARCH

Work relating to this application was supported by a grant from the U.S. Government (GM67101, AI41637, and AI54696). The government may have certain rights in the invention.

### BACKGROUND OF THE INVENTION

The production of pro-inflammatory cytokines such as tumor necrosis factor (TNF) in macrophages is essential for initiating innate immune responses and maintaining the systemic inflammatory state that accompanies diseases such as sepsis (Beutler, Nature 430:257-63 (2004); Cohen, Nature 420:885-91 (2002)). Toll-like receptors (TLRs) are primary innate immune sensors, each responding to specific molecules of microbial origin (Janeway & Medzhitov, Annu. Rev. Immunol. 20:197-216 (2002)). TLR4 is the receptor for the lipopolysaccharide (LPS) endotoxin, and it responds to LPS by recruiting signaling adaptors, such as the myeloid differentiation primary response gene 88 (MyD88) and Toll/interleukin-1 receptor/resistance adaptor protein (TRIF) (also known as TICAM-1). This recruitment allows for the interaction and activation of IRAK family members and TNF receptor-associated factor 6 (TRAF6), which subsequently activate the NF-κB and MAP kinase pathways that control inflammatory cytokine production (Akira & Takeda, Nat. Rev. Immunol. 4:499-511 (2004)).

Although the NF-κB and MAP kinase pathways are activated transiently in macrophages within a few hours of lipopolysacharride (LPS) treatment, the production of pro-inflammatory cytokines like TNF can last up to 24 hours. Thus, the production of inflammatory cytokines is part of an inflammation process that is characterized by an initiation followed by a later “sustained” phase. The sustained phase normally ends when there is a resolution of the inflammatory trigger. Sustained cytokine production is very important in maintaining an inflammatory state and in the development of inflammatory diseases because many of the events culminating in a breakdown in the regulation of inflammation occur near the end of a sustained inflammatory response. Therefore, sustained TNF production is associated with the pathology of many inflammatory diseases.

Thus, an agent that can affect sustained TNF production would be useful for the development of therapeutics for the treatment of inflammatory diseases.

### SUMMARY OF THE INVENTION

The present invention involves the discovery of the role of 4-1BB ligand (4-1BBL) in the production of pro-inflammatory cytokines important in mediating macrophage activation. In particular, the invention involves the discovery that 4-1BBL acts in later phase signaling events that are important for sustained production of tumor necrosis factor (TNF) that results in inflammation. The function of 4-1BBL in later phrase cytokine production is independent of 4-1BB. Thus, the invention relates to a novel function of 4-1BBL that is independent from its known receptor 4-1BB and provides a method for reducing production of an inflammatory cytokine. The invention also provides for 4-1BBL antagonists and blocking agents, as well as pharmaceutical compositions and articles of manufacture comprising such antagonists and blocking agents. In addition, the invention provides methods for screening for 4-1BBL blocking agents and antagonists.

One aspect of the invention is a pharmaceutical composition comprising a 4-1BBL blocking agent and a pharmaceutically acceptable carrier, wherein the 4-1BBL blocking agent is selected from the group consisting of (a) an antagonistic antibody specific for 4-1BBL; (b) an oligonucleotide effective to reduce expression from a 4-1BBL nucleic acid in a cell; and (c) a soluble 4-1BB. A therapeutically effective amount of the blocking agent can be employed in the composition.

One aspect of the invention is a pharmaceutical combination comprising a 4-1BBL blocking agent and a second medicament that is an anti-inflammatory drug, wherein the 4-1BBL blocking agent is selected from the group consisting of (a) an antagonistic antibody specific for 4-1BBL; (b) an oligonucleotide effective to reduce expression from a 4-1BBL nucleic acid in a cell; and (c) a soluble 4-1BB. A therapeutically effective amount of the blocking agent and/or the anti-inflammatory drug can be employed in the composition. The anti-inflammatory drug can be an antibody specific for tumor necrosis factor.

Another aspect of the invention is a chimeric or humanized antibody specific for 4-1BBL.

Another aspect of the invention is a soluble 4-1BB that binds specifically to with a mammalian 4-1BBL. In some embodiments, the mammalian 4-1BBL is that of a human, mouse, rabbit, pig or horse. In some embodiments, the soluble 4-1BB has a sequence that corresponds to amino acid 23 to amino acid 186 of the human 4-1BB polypeptide; amino acid 24 to amino acid 187 of the mouse 4-1BB polypeptide; amino acid 1 to amino acid 186 of the human 4-1BB polypeptide; to amino acid 1 to amino acid 187 of the mouse 4-1BB polypeptide; or a corresponding region in another mammalian 4-1BB. In some embodiment, the soluble 4-1BB has the sequence of SEQ ID NO: 7, 8, 20 or 21.

Another aspect of the invention is an article of manufacture comprising a vessel containing a 4-1BBL blocking agent and instructions for use of the 4-1BBL blocking agent for reducing inflammation and/or reducing production of an inflammatory cytokine. In some embodiments, the instructions for use of the 4-1BBL blocking agent includes instructions for use of the 4-1BBL blocking agent in the treatment of an inflammatory condition. A therapeutically effective amount of the blocking agent can be provided in the vessel.

In some embodiments, the inflammatory condition is rheumatoid arthritis, juvenile rheumatoid arthritis, psoriatic arthritis, psoriasis, ankylosing spondylitis, Crohn's disease, rheumatoid arthritis, systemic-onset juvenile chronic arthritis, osteoporosis, or irritable bowel syndrome.

Another aspect of the invention is a method for reducing production of an inflammatory cytokine in a mammal comprising administering a 4-1BBL blocking agent to the mammal. A therapeutically effective amount of the blocking agent can be administered. In some embodiment, the method further comprises identifying a mammal suffering from or at risk for an inflammatory condition. In some embodiments, the mammal is a human, mouse, rabbit, pig or horse.

Another aspect of the invention is a method for reducing inflammation in a mammal comprising administering a 4-1BBL blocking agent to the mammal. A therapeutically effective amount of the blocking agent can be administered. In some embodiments, the mammal is a human, mouse, rabbit, pig or horse.

Another aspect of the invention is a method of screening for a 4-1BBL blocking agent that involves (a) contacting a cell expressing 4-1BBL with a candidate agent; and (b) determining whether the candidate agent decreases production of an inflammatory cytokine, or whether the candidate agent prevents 4-1BBL oligomerization, wherein the candidate agent is a 4-1BBL blocking agent if it decreases production of an inflammatory cytokine or prevents 4-1BBL oligomerization. In some embodiments, the inflammatory cytokine is tumor necrosis factor or IL-6. In some embodiments, the cell is a human cell, a macrophage, and/or a RAW264.7 cell.

Another aspect of the invention is a method for screening for a 4-1BBL blocking agent that involve (a) administering a candidate agent to a mammal; and (b) determining whether the candidate agent reduces inflammation, or decreases production of an inflammatory cytokine, in the mammal, wherein the candidate agent is a 4-1BBL blocking agent if it reduces inflammation, or decreases production of an inflammatory cytokine, in the mammal. In some embodiments, the mammal has an inflammatory condition. In some embodiments, the inflammatory cytokine is tumor necrosis factor or IL-6. In some embodiments, the production of the inflammatory cytokine is decreased by 20%, 25%, 30% or more than 30%. In some embodiments, the mammal is a mouse, rat, rabbit, or pig.

In some embodiments of the invention, the 4-1BBL is a mammalian 4-1BBL. In some embodiments, the 4-1BBL is that of a human, mouse, rat, rabbit pig or horse. In some embodiments, the 4-1BBL has the sequence set out in SEQ ID NO: 1 or 2. In some embodiments, the blocking agent is a chimeric or a humanized antibody specific for 4-1BBL. In some embodiments, the blocking agent is an oligonucleotide having a sequence selected from the group consisting of SEQ ID NO: 10-15, 22-38, and 45-56. In some embodiments, the blocking agent is a soluble 4-1BB that binds to human 4-1BBL. In some embodiments, the soluble 4-1BB has a sequence corresponding to amino acid 23 to amino acid 186 of the human 4-1BB polypeptide; amino acid 24 to amino acid 187 of the mouse 4-1BB polypeptide; amino acid 1 to amino acid 186 of the human 4-1BB polypeptide; or to amino acid 1 to amino acid 187 of the mouse 4-1BB polypeptide. In some embodiments, the blocking agent is a soluble 4-1BB having the sequence set out in SEQ ID NO: 7, 8, 20 or 21. In some embodiment, the inflammatory cytokine is tumor necrosis factor or IL-6.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents and other references mentioned herein are incorporated by reference in their entirety. Amino acid designations may include full name, three-letter, or single-letter designations as commonly understood by one of ordinary skill in the art to which this invention belongs. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

FIG. 1A-D are results demonstrating that 4-1BBL is a TLR4-interacting protein. (A) Growth of yeast cells transfected with expression plasmids encoding amino acids 653-839 of TLR4 and amino acids 5-254 of 4-1BBL (clone 1), amino acids 3-254 of 4-1BBL (clone 2), dominant negative p38α (AF), p53 or lamin, on medium lacking histidine. 4-1BBL, but not unrelated proteins, interact with the TLR4 intracellular domain. (B) Immunoassay of 293T cells transfected with empty plasmid (Control) or plasmid expressing hemagglutinin-tagged 4-1BBL (HA-4-1BBL) and Flag-tagged TLR4 or IL-3R, lysed 24 hours after transfection; two thirds of the lysates were immunoprecipitated (IP) with anti-hemagglutinin (HA). TLR4, but not IL-3R, was pulled down by 4-1BBL. (C) Immunoassay of 293T cells transfected with plasmids expressing GFP-tagged 4-1BBL and Flag-tagged TLR4, TLR4 lacking the cytosolic domain (TLR4-ΔCyt), TLR4 lacking the extracellular domain (TLR4-ΔExt) or IL-3R (left) or Myc-tagged TLR4 or TLR4 with the P712H substitution (TLR4-P712H; right); lysates were immunoprecipitated with anti-Flag or anti-Myc. The TLR4 intracellular domain is involved in the interaction with 4-1BBL, and distinct portions of the TLR4 Toll-IL-1R domain interacts with 4-1BBL and MyD88. (D) Immunoassay of 293T cells transfected with plasmid expressing Flag-tagged TLR4 and GFP alone or GFP-tagged 4-1BBL, 4-1BBL lacking the cytosolic domain (4-1BBL-ΔCyt), 4-1BBL lacking the extracellular domain (4-1BBL-ΔExt) or 4-1BBL containing the cytosolic domain only (4-1BBL-Cyt). The cytosolic domain of 4-1BBL and association with the cell membrane are required for 4-1BBL to interact with TLR4. IP: immunoprecipitation; IB: immunoblot. Results are representative of two to three independent experiments.

FIG. 2A-D are results demonstrating the in vivo effect of inhibition of 4-1BBL. (A) Survival of wild-type and 4-1BBL-deficient mice injected intraperitoneally with 0.5 mg LPS per 25 g body weight. 4-1BBL knockout (KO) mice were resistant to the lethal effect of LPS. Survival of wild-type mice injected intraperitoneally with 300 μg LPS (B) or 500 μg LPS (C) per 25 g body weight together with isotype control IgG2a or anti-4-1BBL (250 μg per 25 g body weight). The lethal effect of LPS on wild-type mice was attenuated by the administration of anti-4-1BBL. (D) ELISA of TNF in serum from wild-type or 4-1BBL-deficient mice injected with LPS. LPS-induced TNF production was much lower in 4-1BBL KO mice in comparison with wild type mice. Log-rank test was used in (A) and (B). P=0.0017 in (A); P=0.018 in left panel of (B); P=0.048 in right panel of (B). Data in (C) are presented as means±s.d. *, P<0.005, **, P<0.001 (Student's t-test).

FIG. 3A-H are results demonstrating that 4-1BBL is required for sustained TNF production in LPS-stimulated macrophages. (A) ELISA of TNF, IL-6, IL-1β or the p40 chain of IL-12 (1′-12p40) in culture medium of wild-type (WT) or 4-1BBL-deficient (4-1BBL KO) peritoneal macrophages left untreated (None) or treated for 24 hours with LPS (100 ng/mL; LPS). Bottom right, production of IL-6 induced by IL-1β (10 ng/mL; control). 4-1BBL-deficient macrophages produced less TNF, IL-6 and IL-12 than did wild-type macrophages. (B & C) ELISA of TNF production in culture medium of wild-type and 4-1BBL-deficient macrophages (B) or 4-1BB-deficient macrophages (4-1BB KO; C) treated with LPS (time, horizontal axis). 4-1BBL was essential for the TNF production during later times after LPS stimulation, but 4-1BB was not required for the promotion of sustained LPS-induced TNF production. (D) Quantitative PCR of TNF mRNA by wild-type and 4-1BBL-deficient macrophages treated with LPS (time, horizontal axis). AU: arbitrary units. TNF mRNA peaked at similar quantities in 4-1BBL-deficient and wild-type macrophages at two hours after LPS stimulation, but there was much less TNF mRNA in 4-1BBL-deficient cells than in wild-type macrophages at later times after LPS stimulation. (E) RNA dot blot of a nuclear run-on analysis of TNF and glyceraldehyde phosphate dehydrogenase (GAPDH; control) transcription rates in wild-type and 4-1BBL-deficient macrophages stimulated with LPS (100 ng/mL; time, above lanes). LPS triggered Tnf transcription at 1 hour after LPS stimulation in both 4-1BBL-deficient and wild-type macrophages, but Tnf transcription was reduced in 4-1BBL-deficient macrophages at later times after LPS treatment. (F) Real-time PCR analysis of the stability of TNF mRNA in wild-type and 4-1BBL-deficient macrophages at 3 hour after LPS stimulation. Actinomycin D (10 μg/mL) was used to inhibit transcription. Values are the percent remaining relative to the value at 0 hour (set at 100%). The deletion of 4-1BBL had a considerable influence on the stability of TNF mRNA. (G) EMSA of nuclear extracts from wild-type and 4-1BBL-deficient macrophages stimulated with LPS (time, above lanes), assessed with radiolabeled probes (left margin). 4-1BBL deletion had no influence on LPS-induced NF-κB activation or LPS-induced activation of the transcription factor AP-1. (H) Immunoblot of lysates of wild-type or 4-1BBL-deficient macrophages treated with LPS (time, above lanes). NF-κB and MAP kinase pathways are not significantly affected by 4-1BBL deletion. p-, phosphorylated. P<0.01, and P<0.005, versus control (Student's t-test). Data are the mean±s.d. of triplicates (A-D) and are representative of two to three experiments (A-H).

FIG. 4A-D are results demonstrating that 4-1BBL is required for sustained TNF production in RAW264.7 macrophages. (A) RAW 264.7 cells were stably transfected with pSuppressor-containing control siRNA, or 4-1BBL-siRNA#1 or #2. The protein levels of 4-1BBL were measured by immunoblotting. (B) TNF production was measured in control and 4-1BBL knockdown cells treated with LPS (100 ng/mL) for the indicated times. (C) TNF production was measured in control and 4-1BBL knockdown cells treated with peptidoglycan (PG, 10 μg/mL), poly I:C (25 μg/mL), LPS (100 ng/mL), CpG (5 μg/mL), or culture media for 24 hours. Both siRNAs effectively knocked down 4-1BBL expression in RAW264.7 cells (A) and inhibited LPS- and other TLR ligand-induced TNF production (B and C). 4-1BBL has no role in LPS-induced NF-κB activation in RAW264.7 cells as reflected in I-κB-α degradation (D). Data are presented as means±s.d. of three independent experiments done in duplicate. *, P<0.05, and **, P<0.01 versus control. Result in (a) is representative of 3 experiments.

FIG. 5A-B are results demonstrating that 4-1BB is not involved in LPS-induced TNF production in RAW264.7 macrophages. (A) RAW264.7 cells were stably transfected with pSuppressor-containing control siRNA, 4-1BB-siRNA#1 or #2. 4-1BB mRNA was examined by RT-PCR. (B) TNF production was measured in control and 4-1BB knockdown cells after LPS (100 ng/mL) treatment for 24 hours. Small interfering RNA effectively knocked down 4-1BB production in RAW264.7 cells (A), but no effect was observed on LPS-induced TNF production (B). Data are presented as means±s.d. of three independent experiments done in duplicate.

FIG. 6A-D are results demonstrating that 4-1BBL is involved in TLR-ligands-induced TNF production in macrophages. (A) 293T cells were transfected with an empty (control) or HA-4-1BBL expression vectors, together with Flag-TLR2, Flag-TLR3, Flag-TLR4, or Flag-TLR9. The cells were lysed 24 hours after transfection. The cell lysates were immunoblotted with anti-Flag antibodies, or were immunoprecipitated with anti-HA and then immunoblotted with anti-HA and anti-flag antibodies. All TLRs tested were pulled down by 4-1BBL in the co-immunoprecipitation assays. (B) Wildtype and 4-1BBL-deficient macrophages were treated with Pam3 (1 μg/mL), PolyI:C (25 μg/mL), LPS (100 ng/mL), R848 (100 nM), CpG (5 μg/mL), IL-1β (10 ng/mL), or culture media (None). The TNF production was measured after 24 hours of treatment. Pam3 (TLR2 ligand)-, PolyI:C (TLR3 ligand)-, R848 (TLR7&8 ligand)-, CpG (TLR9 ligand)-induced production of TNF were reduced in 4-1BBL KO cells. (C) Wildtype and TLR4-deficient macrophages were treated with 0, 1, 2.5 or 5 μg/mL of CpG DNA and incubated for 24 hours, or (D) treated with 1 μg/mL of CpG DNA and culture supernatant was collected at the indicated time points to measure TNF production. A small but statistically significant reduction in TLR9-induced TNF production in TLR4-deficient macrophages when a low dose of CpG (1 μg/mL) was used. Data are presented as means±s.d. of triplicates. *, P<0.05, **, P<0.01, and ***, P<0.005. Results are representative of two to four experiments.

FIG. 7A-F are results demonstrating that 4-1BBL induction and cell surface location is required for sustained TNF production in LPS-treated macrophages. (A) Immunoblot of 4-1BBL in lysates from wild-type macrophages and macrophages deficient in TLR4 (TLR4 KO), MyD88 (MyD88 KO) or TRIF (TRIF KO), treated with LPS (time, above lanes). LPS induces quick expression of 4-1BBL in macrophages in a TLR4-, MyD88-, and TRIF-dependent manner. (B) Semiquantitative PCR of 4-1BBL mRNA in wild-type macrophages left without pretreatment (None) or pretreated for 1 hour with inhibitors of NF-κB (20 μM sulfasalazine), p38 (10 μM SB203580), Jnk (5 μM SP600125) or MEK (10 μM PD98059) and then treated with LPS (time, above lanes). LPS-induced 4-1BBL mRNA was effectively inhibited by the NF-κB inhibitor sulfasalazine and the proteasome inhibitor MG-132 (data not shown). In addition, the p38 inhibitor SB203580, the Jnk inhibitor SP600125, and the Mek inhibitor PD98059 also had some inhibitory effects on 4-1BBL expression. (C) Real-time RCR analysis of the stability of LPS-induced 4-1BBL mRNA (1 hour of treatment; LPS) or ectopically expressed 4-1BBL mRNA at 18 hour after infection with recombinant adenovirus encoding 4-1BBL (Adv). Actinomycin D was used to inhibit transcription. Values are the percent remaining relative to the value at 0 hour (set at 100%). LPS-induced alterations in mRNA stability were also involved in LPS-induced 4-1BBL expression. (D) Flow cytometry of 4-1BBL expression by wild-type peritoneal macrophages treated with LPS (time, key): right, surface expression; left, total expression in cells made permeable with 0.1% saponin. LPS-induced 4-1BBL is located on the cell surface. (E) Immunofluorescence microscopy of wild-type peritoneal macrophages preincubated for 1 hour with (+) or without (−) brefeldin A (3 μg/mL), then treated with LPS (time, left margin) and immunostained with anti-4-1BBL. Original magnification, ×100. Brefeldin A blocks protein translocation to the cell surface by inhibiting its translocation from the endoplasmic reticulum to the Golgi apparatus. (F) Semiquantitative PCR of 4-1BBL and TNF mRNA in lysates of cells treated as described in E. Brefeldin A neither affected the LPS-induced increase in 4-1BBL mRNA nor influenced LPS-induced TNF mRNA after 1 hour, but did reduce TNF mRNA at 2, 4 and 6 hours. GAPDH (A,B,F): glyceraldehyde phosphate dehydrogenase (loading control). Data are representative of three independent experiments.

FIG. 8 A-F are results indicating that TLR ligands, but not IL-1β, induce 4-1BBL expression in macrophages. Wildtype macrophages were treated with LPS, IL-1β, Poly I:C, peptidoglycan, CpG DNA, or R848 for time periods as indicated. Culture supernatants of cells treated with nothing (None), LPS, or IL-1β for 24 hours were collected and IL-6 levels were measured by ELISA. Data in (B) are presented as means±s.d. of triplicates. 4-1BBL expression was analyzed by Western blotting with anti-4-1BBL antibodies. GAPDH was used as a control. Results are representative of two to three experiments.

FIG. 9A-G are results indicating that the expression and crosslinking of 4-1BBL triggers TNF production. Immunoblot of 4-1BBL (above) and ELISA of TNF production (below) by wild-type macrophages (A), wild-type and 4-1BB-deficient macrophages (B), wild-type and TLR4-deficient macrophages (C), wild-type and TLR2-deficient macrophages (D) or wild-type and TRIF-deficient macrophages (E) infected with various doses (above lanes and horizontal axes) of adenovirus expressing nothing (Control) or 4-1BBL. PFU: plaque-forming units. 4-1BBL expression induced TNF production in a dose-dependent manner (A) that did not require 4-1BB (B). TNF induction is partially impaired in macrophages isolated from TLR4−/− mice (C) and was lower in TLR2-deficient macrophages (D). TNF induction was independent of TRIF as TRIF deficiency had no effect on 4-1BBL-induced TNF production (E). (F) ELISA of TNF production by wild-type macrophages or macrophages deficient in 4-1BBL, MyD88, TRIF, TLR2, TLR4 or both MyD88 and TRIF, treated for 24 hours with goat anti-human Fc (Anti-Fc; 1.5 μg/mL), 4-1BB-Fc (5 μg/mL), both together or culture medium alone (None). Crosslinking of more than two 4-1BBL molecules is needed to trigger TNF production. (G) ELISA of TNF production by wild-type macrophages incubated for 24 hours with culture medium (None) or LPS alone or in combination with Fc, 4-1BB-Fc or anti-4-1BBL (α-4-1BBL; 0, 2 or 5 μg/mL, with isotype antibody IgG2a added to a total of 5 μg/mL each). Both 4-1BB-Fc and anti-4-1BBL antibodies inhibited LPS-induced TNF production. *, P<0.05, **, P<0.01, and ***, P<0.005, versus control (Student's t-test). Data are the mean±s.d. of triplicate samples and are representative of two to four experiments.

FIG. 10A-I indicate that two sequential TLR4 complexes exist. (A) Immunoassay of wild-type macrophages treated with LPS (time, above lanes); total cell lysates (Lysates; top) or lysates immunoprecipitated with anti-TLR4, anti-MyD88 or anti-4-1BBL were analyzed by immunoblot with anti-4-1BBL, anti-TLR4 and/or anti-MyD88. Dashed outline, isotype antibody (negative control for immunoprecipitation); solid outlines, positive control for immunoblot of 4-1BBL or MyD88. In LPS-treated macrophages, the MyD88-TLR4 complex is responsible for the initial cellular responses, and the 4-1BBL-TLR4 complex is involved in sustaining TNF production. (B) Flag-TLR4, but not flag-4-1BBL, was pulled down by MyD88 indicating that MyD88 is not able to interact with 4-1BBL. (C) 4-1BBL interacts with TRAF6, but not TRAF2. (D) Knockdown of TRAF6 impaired LPS, PolyI:C, and IL-1β induced cytokine production, but has no effect on TNF-induced IL-6 production. (E) EMSA of nuclear extracts from wild-type macrophages infected (time, above lanes) with adenovirus expressing GFP (Adv-GFP) or 4-1BBL (Adv-4-1 analyzed with radiolabeled probes (left margin). LPS (bottom right), LPS-treated sample (positive control for NF-κB). Expression of 4-1BBL activated CREB and C/EBP but not NF-κB. (F) Immunoblot of various proteins (left margin) in total lysates of cells treated as described in E. (G) ELISA of TNF production by wild-type macrophages infected with adenovirus expressing 4-1BBL, treated 6 h after infection with inhibitors of NF-κB (20 μM sulfasalazine), p38 (10 μM SB203580), Jnk (5 μM SP600125) or MEK (10 μM PD98059) and analyzed 24 hour after infection. *, P<0.01, and **, P<0.005, versus control (Student's t-test). (H) Top, semiquantitative PCR of TNF mRNA in wild-type macrophages infected with adenovirus expressing 4-1BBL (time, above lanes). Bottom, stability of TNF mRNA in wild-type macrophages infected for 18 hours with adenovirus or treated for 1 hour with LPS. Actinomycin D was used to inhibit transcription. Values are the percent remaining relative to the value at 0 hour (set at 100%). Data represent the mean±s.d. of triplicates (G) and are representative of two (A,G,H), three (E) or four (F) experiments. No degradation of IκBα was observed in cells overexpressing 4-1BBL (F). Expression of 4-1BBL triggered some phosphorylation of p38, Jnk and Erk (F), and inhibition of p38, Jnk and Erk (but not NF-κB) reduced 4-1BBL-mediated TNF production (G). Deletion of 4-1BBL resulted lower LPS-induced to TNF mRNA transcription at later times, while 4-1BBL overexpression resulted in more TNF mRNA (H). TNF transcripts had similar half-lives in cells overexpressing 4-1BBL and LPS-treated cells (H). (I) A model of the sequential signaling in LPS-treated macrophages. TLR4-LPS ligation leads to MyD88- and TRIF-dependent expression of inflammatory genes including TNF within a few hours. 4-1BBL is induced by this early response and translocates to the cell surface where it interacts with TLR and initiates signaling for the sustained expression of inflammatory genes such as TNF.

FIG. 11A-F summarizes data illustrating the involvement of 4-1BBL in IL-6 induction. 4-1BBL−/− mice produce significantly less IL-6 in response to LPS than wild type mice (A). Macrophages from wild type mice produced significantly more IL-6 than macrophages from 4-1BBL−/− mice (B). Wild type macrophages treated with Pam3, PolyI:C, LPS, R848, and CpG produced significantly more IL-6 than 4-1BBL−/− macrophages treated with the same inducers (C). Macrophages incubated with LPS and 4-1BB-Fc produced significantly less IL-6 than macrophages incubated with LPS alone, and macrophages incubated with LPS and increasing concentrations of anti-4-1BBL antibodies exhibited decreasing levels of IL-6 production (D). RAW264.7 cells in which 4-1BBL expression is reduced by 4-1BBL-specific siRNA produces significantly less IL-6 than RAW264.7 cells treated with control siRNA (E). Cells in which 4-1BBL expression is reduced by 4-1BBL-specific siRNA and treated with peptidoglycan (PG, 10 μg/mL), PolyI:C (25 μg/mL), LPS (100 ng/mL), CpG (5 μg/mL) produce significantly less IL-6 than cells transfected with control siRNA and treated with peptidoglycan (PG, 10 μg/mL), Polyl:C (25 μg/mL), LPS (100 ng/mL), CpG (5 μg/mL) (FIG. 11F).

FIG. 12 is a schematic diagram illustrating the structural domains of mouse 4-1BBL (A) and human 4-1BBL (B). “Cyt” indicates cytoplasmic domain, “TM” indicates transmembrane domain, “Ext” indicates extracellular domain and “Sig” indicates signal peptide.

FIG. 13 is a schematic diagram illustrating the structural domains of mouse 4-1BB (A), human 4-1BB (B), and soluble mouse 4-1BB-human Ig-Fc fusion protein (C). “Cyt” indicates cytoplasmic domain, “TM” indicates transmembrane domain, “Ext” indicates extracellular domain and “Sig” indicates signal peptide.

### DETAILED DESCRIPTION OF THE INVENTION

The present invention involves the discovery of the role of 4-1BB ligand (4-1BBL) in the production of pro-inflammatory cytokines in macrophage activation. In particular, the invention involves the discovery that 4-1BBL acts in later phase signaling events that result in sustained production of inflammatory cytokines such as TNF leading to over-sustained inflammation. The function of 4-1BBL in later phrase signaling is independent of 4-1BB. Thus, the invention provides for 4-1BBL blocking agents, as well as pharmaceutical compositions and articles of manufacture comprising such blocking agents that can be used in methods for reducing production of inflammatory cytokine such as TNF and IL-6 in a 4-1BB-independent manner. The invention provides methods for reducing inflammation, methods for reducing production of inflammatory cytokine, as well as methods for screening for 4-1BBL blocking agents.

4-1BBL Polypeptides and Nucleic Acids

The studies described herein show that initial induction of TNF expression and sustained TNF production in macrophages are regulated by early and later phase signaling events. Toll-like receptor (TLR) 4-mediated TNF production in macrophages generally occurs within a few hours and is sustained for almost a day (see Galanos et al. Proc. Natl. Acad. Sci. USA 76: 5939-43 (1979). Sustained TNF production involves later TLR-signaling that is controlled by the cell surface 4-1BB ligand (4-1BBL). The 4-1BBL functions independently of the myeloid differentiation primary response gene 88 (MyD88) and the Toll/interleukin-1 receptor/resistance adaptor protein (TRIF), but is dependent on TNF receptor-associated factor 6 (TRAF6). Thus, the signal some TLR4/MyD88/TRIF is responsible for the initiation and early phase expression of inflammatory genes, while the 4-1BBL is responsible for the later phase of sustained TNF production. The 4-1BBL is one of the early induced proteins in macrophage in response to inflammatory stimuli and is only induced in the early phase of macrophage activation. The newly synthesized 4-1BBL translocates onto the cell surface to generate a new phase of signaling for the later phase sustained TNF production. Thus, diseases associated with over production of an inflammatory cytokine such as, for example, TNF or IL-6, or an otherwise break down in the regulation of sustained inflammatory responses can be treated with agents that can inhibit or reduce the activity of a 4-1BBL polypeptide or the expression from a 4-1BBL nucleic acid.

4-1BBL (4-1BB ligand) is a member of the TNF family of type 11 cell surface glycoprotein expressed on activated antigen presenting cells such as activated B cells and macrophages. 4-1BBL polypeptides are known in the art and can be from any source, for example, from a mouse, a rabbit, a pig, a dog, a cow, a monkey, or a human. An example of a mouse 4-1BBL polypeptide is the following (GenBank Accession number NP_{—}033430):

```
(SEQ ID NO: 1)
    1 MDQHTLDVED TADARHPAGT SCPSDAALLR OTGLLADAAL LSDTVRPTNA
  51 ALPTDAAYPA VNVRDREAAW PPALNFCSRH PKLYGLVALV LLLLIAACVP
101 IFTRTEPRPA LTITTSPNLG TRENNADQVT PVSHIGCPNT TQQGSPVFAK
151 LLAKNQASLC NTTLNWHSQD GAGSSYLSQG LRYEEDKKEL VVDSPGLYYV
201 FLELKLSPTF TNTGHKVQGW VSLVLQAKPQ VDDFDNLALT VELFPCSMEN
251 KLVDRSWSQL LLLKAGHRLS VGLRAYLHGA QDAYRDWELS YPNTTSFGLF
301 LVKPDNPWE
```

An example of a human 4-1BBL polypeptide is the following (GenBank Accession number P41273):

```
(SEQ ID NO: 2)
    1 MEYASDASLD PEAPWPPAPR ARACRVLPWA LVAGLLLLLL LAAACAVFLA
  51 CPWAVSGARA SPGSAASPRL REGPELSPDD PAGLLDLRQG MFAQLVAQNV
101 LLIDGPLSWY SDPGLAGVSL TGGLSYKEDT KELVVAKAGV YYVFFQLELR
151 RVVAGEGSGS VSLALHLQPL RSAAGAAALA LTVDLPPASS EARNSAFGFQ
201 GRLLHLSAGQ RLGVHLHTEA RARHAWQLTQ GATVLGLFRV TPEIPAGLPS
251 PRSE
```

4-1BBL can be identified based on function or sequence. For example, although the invention is based, in part, on the discovery that in macrophages, 4-1BBL-mediates TNF production in a manner that is independent of 4-1BB, 4-1BBL is also known to interact with 4-1BB, a member of the TNF receptor superfamily of transmembrane proteins expressed on activated CD4 and CD8 T cells to produce a costimulatory signal during T cell-receptor activation. Watts, Annu. Rev. Immunol. 23:23-68 (2005). In addition, the functional domains of human and mouse 4-1BBL are illustrated FIG. 12.

“4-1BBL” or “4-1BBL polypeptide,” as used herein, includes any biologically active fragment of the full-length polypeptide, as well as any fragment that is suitable for use as an immunogen to raise antibody directed to a biologically active 4-1BBL. Thus, a 4-1BBL polypeptide may have an amino acid sequence that is substantially identical to the sequence set out in SEQ ID NO: 1 or 2. In general, the term “substantially identical” means an amino acid sequence differs from the reference sequence only by conservative amino acid substitutions, or example, substitution of one amino acid for another of the same class (e.g. valine for glycine, argine for lysine) or by one or more non-conservative substitutions, deletions or insertions located at positions of the amino acid sequence which do no destroy the function of the protein. A polypeptide (or nucleic acid) sequence is substantially identical to a reference sequence if it exhibits about 50% homology, e.g. 55%, 60%, 65%, 70%, 75%, 80%, 85%, 90%, 95% or more than 95% homology to the reference sequence. A 4-1BBL polypeptide, for example, may be at least about 50%, 55%, 60%, 65%, 70%, 75%, 80%, 85%, 90% or more than 90% identical to SEQ ID NO: 1 and 2.

A 4-1BBL nucleic acid is a polymer of DNA or RNA that encodes a 4-1BBL polypeptide. A 4-1BBL nucleic acid may be genomic DNA as well as messenger RNA. It may be incorporated into a plasmid vector or viral DNA. It may be single strand or double strand, circular or linear. Examples of 4-1BBL nucleic acids include, without limitation, those that encode the mouse and human 4-1BBL polypeptide sequences set forth in SEQ ID NO. 1 and 2. An example of a 4-1BBL nucleic acid that encodes the mouse 4-1BBL polypeptide (SEQ ID NO: 1) is the following sequence which can be found in GenBank under Accession Number NM_{—}009404:

```
(SEQ ID NO: 3)
    1 atggaccagc acacacttga tgtggaggat accgcggatg ccagacatcc
  51 agcaggtact tcgtgcccct cggatgcggc gctcctcaga gataccgggc
101 tcctcgcgga cgctgcgctc ctctcagata ctgtgcgccc cacaaatgcc
151 gcgctcccca cggatgctgc ctaccctgcg gttaatgttc gggatcgcga
201 ggccgcgtgg ccgcctgcac tgaacttctg ttcccgccac ccaaagctct
251 atggcctagt cgctttggtt ttgctgcttc tgatcgccgc ctgtgttcct
301 atcttcaccc gcaccgagcc tcggccagcy ctcacaatca ccacctcgcc
351 caacctgggt acccgagaga ataatgcaga ccaggtcacc cctgtttccc
401 acattggctg ccccaacact acacaacagg gctctcctgt gttcgccaag
451 ctactggcta aaaaccaagc atcgttgtgc aatacaactc tgaactggca
501 cagccaagat ggagctggga gctcatacct atctcaaggt ctgaggtacg
551 aagaagacaa aaaggagttg gtggtagaca gtcccgggct ctactacgta
601 tttttggaac tgaagctcag tccaacattc acaaacacag gccacaaggt
651 gcagggctgg gtctctcttg ttttgcaagc aaagcctcag gtagatgact
701 ttgacaactt ggccctgaca gtggaactgt tcccttgctc catggagaac
751 aagttagtgg accgttcctg gagtcaactg ttgctcctga aggctggcca
801 ccgcctcagt gtgggtctga gggcttatct gcatggagcc caggatgcat
851 acagagactg ggagctgtct tatcccaaca ccaccagctt tggactcttt
901 cttgtgaaac ccgacaaccc atgggaatga
```

An example of a 4-1BBL nucleic acid that encodes the human 4-1BBL polypeptide (SEQ ID NO: 2) is the following sequence which can be found in GenBank under Accession Number U03398:

```
(SEQ ID NO: 4)
      1 gtcatggaat acgcctctga cgcttcactg gaccccgaag ccccgtggcc
    51 tcccgcgccc cgcgctcgcg cctgccgcgt actgccttgg gccctggtcg
  101 cggggctgct gctgctgctg ctgctcgctg ccgcctgcgc cgtcttcctc
  151 gcctgcccct gggccgtgtc cggggctcgc gcctcgcccg gctccgcggc
  201 cagcccgaga ctccgcgagg gtcccgagct ttcgcccgac gatcccgccg
  251 gcctcttgga cctgcggcag ggcatgtttg cgcagctggt ggcccaaaat
  301 gttctgctga tcgatgggcc cctgagctgg tacagtgacc caggcctggc
  351 aggcgtgtcc ctgacggggg gcctgagcta caaagaggac acgaaggagc
  401 tggtggtggc caaggctgga gtctactatg tcttctttca actagagctg
  451 cggcgcgtgg tggccggcga gggctcaggc tccgtttcac ttgcgctgca
  501 cctgcagcca ctgcgctctg ctgctggggc cgccgccctg gctttgaccg
  551 tggacctgcc acccgcctcc tccgaggctc ggaactcggc cttcggtttc
  601 cagggccgct tgctgcacct gagtgccggc cagcgcctgg gcgtccatct
  651 tcacactgag gccagggcac gccatgcctg gcagcttacc cagggcgcca
  701 cagtcttggg actcttccgg gtgacccccg aaatcccagc cggactccct
  751 tcaccgaggt cggaataacg cccagcctgg gtgcagccca cctggacaga
  801 gtccgaatcc tactccatcc ttcatggaga cccctggtgc tgggtccctg
  851 ctgctttctc tacctcaagg ggcttggcag gggtccctgc tgctgacctc
  901 cccttgagga ccctcctcac ccactccttc cccaagttgg accttgatat
  951 ttattctgag cctgagctca gataatatat tatatatatt atatatatat
1001 atatatttct atttaaagag gatcctgagt ttgtgaatgg acttttttag
1051 aggagttgtt ttgggggggg ggtcttcgac attgccgagg ctggtcttga
1101 actcctggac ttagacgatc ctcctgcctc agcctcccaa gcaactggga
1151 ttcatccttt ctattaattc attgtactta tttgcctatt tgtgtgtatt
1201 gagcatctgt aatgtgccag cattgtgccc aggctagggg gctatagaaa
1251 catctagaaa tagactgaaa gaaaatctga gttatggtaa tacgtgagga
1301 atttaaagac tcatccccag cctccacctc ctgtgtgata cttgggggct
1351 agcttttttc tttctttctt ttttttgaga tggtcttgtt ctgtcaacca
1401 ggctagaatg cagcggtgca atcatgagtc aatgcagcct ccagcctcga
1451 cctcccgagg ctcaggtgat cctcccatct cagcctCtcg agtagctggg
1501 accacagttg tgtgccacca cacttggcta actttttaat ttttttgcgg
1551 agacggtatt gctatgttgc caaggttgtt tacatgccag tacaatttat
1601 aataaacact catttttcc
```

A 4-1BBL nucleic acid may also encode a fragment of the polypeptide sequences set forth in SEQ ID NO: 1 and 2 provided that the nucleic acid encodes a biologically active polypeptide or fragment that is suitable for use as an immunogen to raise 4-1BBL specific antibody as discussed above.

4-1BBL Blocking Agents Generally

A 4-1BBL blocking agent may be a macromolecule or a small molecule that inhibits or reduces the expression and/or activity of 4-1BBL. A 4-1BBL blocking agent can inhibit or reduce the activity of 4-1BBL. Examples of such 4-1BBL blocking agents include, without limitation, a 4-1BBL-specific antibody, a soluble form of 4-1BB, and an agent or small molecule that interferes with protein translocation to the cell surface such as Brefeldin A. These 4-1BBL blocking agents act by interfering with 4-1BBL oligomerization and/or interfere with its interaction with the appropriate signaling molecules, e.g. TLRs and TRAF6, in the later phase signaling events involved in TNF production. The term “4-1BBL oligomerization” as used herein, refers to the formation of an aggregate of 4-1BBL molecules that is required for 4-1BBL-mediated production of inflammatory cytokines such as, for example, TNF or IL-6. 4-1 oligomerization refers to the formation of an aggregate of more than two 4-1BBL molecules, e.g. an aggregate of three or four 4-1BBL molecules. A 4-1BBL blocking agent may also interfere with protein translocation to the cell surface as cell surface localization of 4-1BBL is required for the proper functioning during the later signaling phase.

A 4-1BBL blocking agent can also act by inhibiting or reducing expression from a 4-1BBL nucleic acid. A 4-1BBL blocking agent can act at the transcriptional or translational level such that the amount of 4-1BBL produced by the cell is altered. A 4-1BBL blocking agent can decrease the production of 4-1BBL mRNA transcripts or decrease the stability of the mRNA transcripts. These blocking agents include, without limitation, an oligonucleotide such as an antisense RNA, an siRNA and a ribozyme. Such oligonucleotide-based 4-1BBL blocking agent can hybridize to a 4-1BBL nucleic acid under physiological (e.g. about 37° C., pH 7 to 7.8, and physiological concentrations of electrolyte) or at stringent or highly stringent conditions and inhibit or reduce expression from the 4-1BBL nucleic acid. These blocking agents may also include inhibitors of other molecules that are involved in 4-1BBL expression or activity.

An oligonucleotide is a polymer of ribose nucleotides or deoxyribose nucleotides having more than 3 nucleotides in length. An oligonucleotide may include naturally-occurring nucleotides; synthetic, modified, or pseudo-nucleotides such as phosphorothiolates; as well as nucleotides having a detectable label such as P³², biotin or digoxigenin.

An oligonucleotide that can reduce the expression of a 4-1BBL nucleic acid, that is an oligonucleotide of the invention, may be completely complementary to the 4-1BBL nucleic acid. Alternatively, some variability between the sequences may be permitted. An oligonucleotide that can hybridize to a 4-1BBL nucleic acid under physiological conditions, for example, physiological temperatures and salt concentrations, or under stringent or highly stringent hybridization conditions, is sufficiently complementary to inhibit expression of a 4-1BBL nucleic acid. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a 4-1BBL coding sequence, each separated by a stretch of contiguous nucleotides that are not complementary to adjacent coding sequences, may inhibit the function of a 4-1BBL nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an oligonucleotide hybridized to a sense nucleic acid to estimate the degree of mismatching that will be tolerated for inhibiting expression of a particular target nucleic acid. An oligonucleotide-based 4-1BBL blocking agent of the invention include, for example, a small interfering RNA (siRNA), an antisense nucleic acid or a ribozyme.

A 4-1BBL blocking agent inhibits or reduces the expression and/or activity of 4-1BBL by any amount such as, for example, by 2%, 5%, 10%, 20%, 40%, 60%, 80%, 95% or 100%. The activity of 4-1BBL can be determined using methods known in the art including the methods describe herein such as, without limitation, assaying for sustained TNF production, determining whether 4-1BBL interacts with TLRs or TRAF6, for example, and determining whether 4-1BBL is at the cell surface. The expression of 4-1BBL can be determined also using methods known in the art including, without limitation, northern hybridization to determine the level of 4-1BBL transcript or western hybridization to determine the level of 4-1BBL polypeptide.

Examples of various types of 4-1BBL blocking agents are discussed in detailed below.

4-1BBL-Specific Antibody

A 4-1BBL blocking agent can be an antagonistic antibody directed against a 4-1BBL polypeptide. The term “antibody” refers to an immunoglobin molecule, e.g. a monoclonal antibody, and an immunologically active portion of an immunoglobulin molecule. A monoclonal antibody is a population of antibody molecules that binds specifically with a particular antigen epitope. Immunologically active portions of immunoglobulin molecules include F(ab) and F(ab′)₂ fragments. An anti-4-1BBL antibody can be, without limitation, a murine, chimeric, humanized and/or fully human antibody. A murine antibody is an antibody derived entirely from a murine source, for example, an antibody derived from a murine hybridoma generated from the fusion of a mouse myeloma cell and a mouse B-lymphocyte cell. A chimeric antibody is an antibody that has variable regions derived from a non-human source, e.g. murine or primate, and constant regions derived from a human source. A humanized antibody has antigen-binding regions, e.g. complementarity-determining regions, derived from a mouse source, and the remaining variable regions and constant regions derived from a human source. A fully human antibody is antibody from human cells or derived from transgenic mice carrying human antibody genes.

An antibody directed against 4-1BBL polypeptide is a 4-1BBL-specific antibody, and as such, it will bind to a 4-1BBL polypeptide without binding to a polypeptide that is not 4-1BBL. A 4-1BBL-specific antibody is an antagonist antibody in that it interferes with or blocks proper 4-1BBL function. For example, see FIG. 2A-D. An antagonist antibody may, for example, interfere with oligomerization of more than two 4-1BBL or blocks binding to downstream signaling molecules involved in sustained TNF production, e.g. TLRs or TRAF6, such that production of inflammatory cytokine is reduced. For example, such an antagonist antibody may bind with two 4-1BBL and prevent the formation of an aggregate of more than two 4-1BBL that is needed to mediate cytokine production. Such an antagonist antibody may also bind to 4-1BBL in a region that precludes binding to downstream signaling molecules such as, for example, TLR2, TLR3, TLR4, TLR7, TLR8, TLR9 and TRAF6 that are needed to mediate cytokine production.

Whether the anti-4-1BBL antibody is an antagonist antibody can be determined using methods described herein. For example, the production of inflammatory cytokine such as TNF can be determined in the presence or absence of the antibody. An antibody is an antagonist antibody if its presence results in a decrease in level or duration of inflammatory cytokine production.

Methods to generate antibodies are well known in the art. For example, a 4-1BBL polyclonal antibody can be prepared by immunizing a suitable mammal with an isolated 4-1BBL polypeptide or an antigenic fragment of the 4-1BBL polypeptide. The mammal can be, for example, a rabbit, goat, or mouse. The 4-1BBL polypeptide or antigenic fragment may be expressed using recombinant DNA technology, prepared by chemical synthesis, or purified using standard protein purification techniques. At the appropriate time after immunization, antibody molecules can be isolated from the mammal, e.g. from the blood or other fluid of the mammal, and further purified using standard techniques that include, without limitation, precipitation using ammonium sulfate, gel filtration chromatography, ion exchange chromatography or affinity chromatography using protein A. In addition, the antibody-producing cells of the mammal can be isolated and used to prepare hybridoma cells that secrete 4-1BBL monoclonal antibodies. Techniques for preparing monoclonal antibody-secreting hybridoma cells are known in the art. See, for example, Kohler and Milstein, Nature 256:495-97 (1975) and Kozbor et al. Immunol Today 4: 72 (1983). 4-1BBL monoclonal antibodies can also be prepared using other methods known in the art, such as, for example, expression from a recombinant DNA molecule, or screening of a recombinant combinatorial immunoglobulin library using 4-1BBL polypeptide.

Methods to generate chimeric and humanized monoclonal antibodies are also well known in the art and include, for example, methods involving recombinant DNA technology. A chimeric antibody can be produced by expression from a nucleic acid that encodes a non-human variable region and a human constant region of an antibody molecule. See, for example, Morrison et al., Proc. Nat. Acad. Sci. U.S.A. 86: 6851 (1984). A humanized antibody can be produced by expression from a nucleic acid that encodes a non-human antigen-binding regions (complementarity-determining regions) and a human variable region (without antigen-binding regions) and human constant regions. See, for example, Jones et al., Nature 321:522-24 (1986); and Verhoeven et al., Science 239:1534-36 (1988). Completely human antibodies can be produced by immunizing engineered transgenic mice that express only human heavy and light chain genes. In this case, therapeutically useful monoclonal antibodies can then be obtained using conventional hybridoma technology. See, for example, Lonberg & Huszar, Int. Rev. Immunol. 13:65-93 (1995). Nucleic acids and techniques involved in design and production of antibodies are well known in the art. See, for example, Batra et al., Hybridoma 13:87-97 (1994); Berdoz et al., PCR Methods Appl. 4: 256-64 (1995); Boulianne et al. Nature 312:643-46 (1984); Carson et al., Adv. Immunol. 38:274-311 (1986); Chiang et al., Biotechniques 7:360-66 (1989); Cole et al., Mol. Cell. Biochem. 62:109-20 (1984); Jones et al., Nature 321: 522-25 (1986); Larrick et al., Biochem Biophys. Res. Commun. 160:1250-56 (1989); Morrison, Annu. Rev. Immunol. 10:239-65 (1992); Morrison et al., Proc. Nat'l Acad. Sci. USA 81: 6851-55 (1984); Orlandi et al., Pro. Nat'l Acad. Sci. U.S.A. 86:3833-37 (1989); Sandhu, Crit. Rev. Biotechnol. 12:437-62 (1992); Gavilondo & Larrick, Biotechniques 29: 128-32 (2000); Huston & George, Hum. Antibodies. 10:127-42 (2001); Kipriyanov & Le Gall, Mol. Biotechnol. 26: 39-60 (2004).

Soluble 4-1BB

A 4-1BBL blocking agent also can be a soluble 4-1BB molecule. 4-1BB is a member of the TNF receptor family of type I transmembrane proteins expressed on activated T cells. See Watts, Annu. Rev. Immunol. 23:23-68 (2005). An example of a mouse 4-1BB is the following sequence, which can be found in GenBank under Accession Number NP_{—}035742:

```
(SEQ ID NO: 5)
    1 MGNNCYNVVV IVLLLVGCEK VGAVQNSCDN CQPGTFCRKY NPVCKSCPPS
  51 TFSSIGGQPN CNICRVCAGY FRFKKFCSST HNAECECIEG FHCLGPQCTR
101 CEKDCRPGQE LTKQGCKTCS LGTFNDQNGT GVCRPWTNCS LDGRSVLKTG
151 TTEKDVVCGP PVVSFSPSTT ISVTPEGGPG GHSLQVLTLF LALTSALLLA
201 LIFITLLFSV LKWIRKKFPH IFKQPFKKTT GAAQEEDACS CRCPQEEEGG
251 GGGYEL
```

An example of a human 4-1BB is the following sequence, which can be found in GenBank under Accession Number NP 001552:

```
(SEQ ID NO: 6)
    1 MGNSCYNIVA TLLLVLNFER TRSLQDPCSN CPAGTFCDNN RNQICSPCPP
  51 NSFSSAGGQR TCDICRQCKG VFRTRKECSS TSNAECDCTP GFHCLGAGCS
101 MCEQDCKQGQ ELTKKGCKDC CFGTFNDQKR GICRPWTNCS LDGKSVLVNG
151 TKERDVVCGP SPADLSPGAS SVTPPAPARE PGHSPQIISF FLALTSTALL
201 FLLFFLTLRF SVVKRGRKKL LYIFKQPFNR PVQTTQEEDG CSCRFPEEEE
251 GGGEL
```

4-1BB polypeptides typically include a signal sequence, an extracellular domain, a transmembrane domain, and a cytoplasmic domain as shown in FIG. 13 for the mouse and human polypeptides.

Soluble 4-1BB refers to a 4-1BB polypeptide that is not attached, as a transmembrane protein, to the cell from which it is produced and can inhibit and/or reduce the activity of 4-1BBL. For example, a soluble 4-1BB can be a single polypeptide that consist of the extracellular domain of the full-length 4-1BB. As used herein, the “full-length 4-1BB” is the polypeptide expressed by a cell that has an endogeneous nucleic acid encoding it. The full-length 4-1BB will have an extracellular domain, a transmembrane domain and a cytoplasmic domain. It may or may not have a signal peptide. In contrast, a soluble 4-1BB is any form of 4-1BB other than the full-length as long as it is not attached to a cell as a transmembrane protein. Examples of a soluble 4-1BB polypeptide is a polypeptide corresponding to the extracellular domain of the full-length polypeptide or a fusion polypeptide consisting of the extracellular domain of 4-1BB fused at its C-terminus with an unrelated polypeptide segment such as the Fc fragment of an immunoglobulin molecule, for example, a IgG₁ Fc fragment, or any conventional proteinaceous tag or fusion moiety such as GFP, HA, and FLAG and GST. A soluble 4-1BB can be a single polypeptide or a dimer such as the mouse 4-1BB/Fc discussed the Examples. An example of a soluble 4-1BB that is also a dimer is the mouse 4-1BB/humanIg-Fc polypeptide discussed in the Examples section.

An example of a soluble 4-1BB polypeptide is the extracellular domain of mouse 4-1BB having the following sequence:

```
(SEQ ID NO: 7)
  23 VQNSCDN CQPGTFCRKY NPVCKSCPPS
  51 TFSSIGGQPN CNICRVCAGY FRFKKFCSST HNAECECIEG FHCLGPQCTR
101 CEKDCRPGQE LTKQGCKTCS LGTFNDQNGT GVCRPWTNCS LDGRSVLKTG
151 TTEKDVVCGP PVVSFSPSTT ISVTPEGGPG GHSLQV
```

Another example of a soluble 4-1BB polypeptide is the extracellular domain of human 4-1BB having the following sequence:

```
(SEQ ID NO: 8)
                                                        SLQDPCSN CPAGTFCDNN RNQICSPCPP
  51 NSFSSAGGQR TCDICRQCKG VFRTRKECSS TSNAECDCTP GFHCLGAGCS
101 MCEQDCKQGQ ELTKKGCKDC CFGTFNDQKR GICRPWTNCS LDGKSVLVNG
151 TKERDVVCGP SPADLSPGAS SVTPPAPARE PGHSP
```

Other examples of soluble 4-1BB polypeptide include the first 186 amino acids of the mouse 4-1BB or the first 185 amino acids of the human 4-1BB shown in SEQ ID NO: 20 and 21, respectively.

```
(SEQ ID NO: 20)
    1 MGNNCYNVVV IVLLLVGCEK VGAVQNSCDN CQPGTFCRKY NPVCKSCPPS
  51 TFSSIGGQPN CNICRVCAGY FRFKKFCSST HNAECECIEG FHCLGPQCTR
101 CEKDCRPGQE LTKQGCKTCS LGTFNDQNGT GVCRPWTNCS LDGRSVLKTG
151 TTEKDVVCGP PVVSFSPSTT ISVTPEGGPG GHSLQV
(SEQ ID NO: 21)
    1 MGNSCYNIVA TLLLVLNEER TRSLQDPCSN CPAGTFCDNN RNQICSFCPP
  51 NSFSSAGGQR TCDICRQCKG VFRTRKECSS TSNAECDCTP GFHCLGAGCS
101 MCEQDCKQGQ ELTKKGCKDC CFGTFNDQKR GICRPWTNCS LDGKSVLVNG
151 TKERDVVCGP SPADLSPGAS SVTPPAPARE PGHSP
```

A soluble 4-1BB polypeptide could also have one or more additional amino acids at the N or C terminus of SEQ ID NO: 7, 8, 20 or 21 so long as the polypeptide is able to inhibit or reduce the activity of 4-1BBL, and if the polypeptide is produced by a cell, it is not incorporated into the membrane of the cell as a transmembrane protein. For example, a soluble 4-1BB polypeptide could be a fusion protein consisting of SEQ ID NO: 7, 8, 20 or 21 fused to, for example, glutathione S transferase (GST), the Fc portion of human IgG, a histidine tag, or any unrelated amino acid sequences as discussed above. A soluble 4-1BB polypeptide can also be fragments of the extracellular domain, for example, fragments of SEQ ID NO: 7, 8, 20 and 21 so long as the fragments are biologically active, that is the fragments are able to inhibit or reduce 4-1BBL activity. Methods to determine whether the fragments have biological activity, that is, whether the fragments can inhibit or reduce 4-1BBL activity include, without limitations, those discussed and exemplified herein, for example, methods for determining inflammatory cytokine production by cells such as macrophages in response to LPS and the presence of

4-1BB, a soluble 4-1BB, and biologically active fragments thereof, can be from any source. For example, these can be isolated from a mouse, a rabbit, a pig, a dog, a cow, a monkey, or a human. They can be expressed from a recombinant nucleic acid in a host cell such as, for example, CHO, S2 and E. coli, or a host animal such as, for example, a pig or a monkey or a rabbit, and then isolated using protein purification techniques known to those skilled in the art. Alternatively, they can be synthesized by convention methods of peptide synthesis. Soluble 4-1BB can be obtained from a preparation of the full-length polypeptide by peptidase digestion. Methods for preparing fusion proteins such as that for the mouse 4-1BB-human Ig-polypeptide are well known in the art and include, for example, methods involving recombinant DNA technology. More specifically, a nucleic acid sequence coding for the relevant 4-1BB domains can be ligated to a nucleic acid that encodes the Fc fragment and then expressed in a suitable host cell. Many expression vectors already encoding a fusion moiety and into which a nucleic acid encoding 4-1BB or a biologically active fragment thereof may be cloned are available commercially. The soluble 4-1BB we used is bought from a company.

4-1BBL-Specific Antisense RNA

An antisense RNA may be used to specifically reduce expression of 4-1BBL, for example, by inhibiting transcription and/or translation. An antisense RNA is complementary to a sense nucleic acid encoding 4-1BBL. For example, it may be complementary to the coding strand of a double-stranded cDNA molecule or complementary to an mRNA sequence. It may be complementary to an entire coding strand or to only a portion thereof. It may also be complementary to all or part of the noncoding region of a nucleic acid encoding 4-1BBL. The non-coding region includes the 5′ and 3′ regions that flank the coding region, for example, the 5′ and 3′ untranslated sequences. An antisense RNA is generally at least six nucleotides in length, but may be about 8, 12, 15, 20, 25, 30, 35, 40, 45, or 50 nucleotides long. Longer oligonucleotides may also be used. An antisense RNA may be prepared using methods known in the art, for example, by expression from an expression vector encoding the antisense RNA or from an expression cassette. Alternatively, it may be prepared by chemical synthesis using naturally-occurring nucleotides or modified nucleotides designed to increase biological stability of the RNA or to increase physiological stability of the duplex formed between the antisense RNA and the sense nucleic acid. Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethyl guanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine. Thus, an antisense RNA of the invention may include modified nucleotides as well as natural nucleotides, and it may be any length discussed above that is complementary the sequences provided in SEQ ID NO: 3 and 4.

4-1BBL-Specific SiRNA

Small interfering RNAs may be used to specifically reduce 4-1BBL translation such that the level of 4-1BBL polypeptide is reduced. SiRNAs mediate post-transcriptional gene silencing in a sequence-specific manner. See, for example, http://www.ambion.com/techlib/hottopics/rnai/rnai_may2002_print.html (last retrieved May 10, 2006). Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the G protein mRNA transcript. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003). Typically, a target site that begin with AA, have 3′ UU overhangs for both the sense and antisense siRNA strands, and have an approximate 50% G/C content is selected. SiRNAs may be chemically synthesized, created by in vitro transcription, or expressed from an siRNA expression vector or a PCR expression cassette. See, e.g., http://www.ambion.com/techlib/tb/tb_{—}506html (last retrieved May 10, 2006). When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA (SEQ ID NO: 9). SiRNAs also may be produced in vivo by cleavage of double-stranded RNA introduced directly or via a transgene or virus. Amplification by an RNA-dependent RNA polymerase may occur in some organisms.

Examples of siRNA sequences that can hybridize to a 4-1BBL mRNA transcript include the following sequences and their complementary sequences:

| SEQ ID NO. | Sequence |
|---|---|
| 10 | AAGACGGCGCAGGCGGCAGCG |
| 11 | AAGAGGCCGGCGGGATCGTCG |
| 12 | ATAGTAGACTCCAGCCTTGGC |
| 13 | ATTCCGACCTCGGTGAAGGG |
| 22 | AAGAGGCCGGCGGGAUCGUCG |
| 23 | AUAGUAGACUCCAGCCUUGGC |
| 24 | AUUCCGACCUCGGUGAAGGG |

Their complementary sequences are:

| SEQ ID NO. | Sequence |
|---|---|
| 25 | CGCTGCCGCCTGCGCCGTCTT |
| 26 | CGACGATCCCGCCGGCCTCTT |
| 27 | GCCAAGGCTGGAGTCTACTAT |
| 28 | CCCTTCACCGAGGTCGGAAT |
| 29 | CGCUGCCGCCUGCGCCGUCUU |
| 30 | CGACGAUCCCGCCGGCCUCUU |
| 31 | GCCAAGGCUGGAGUCUACUAU |
| 32 | CCCUUCACCGAGGUCGGAAU |

Small interfering RNA targeting mouse 4-1BBL also include:

| | |
|---|---|
| 5′-GCTCTATGGCCTAGTCGCT-3′; | (SEQ ID NO: 14) |
| 5′-GCAAAGCCTCAGGTAGATG-3′ | (SEQ ID NO: 15) |
| 5′-GCUCUAUGGCCUAGUCGCU-3′ | (SEQ ID NO: 33) |
| 5′-GCAAAGCCUCAGGUAGAUG-3′ | (SEQ ID NO: 34) |

Their complementary sequences are:

| | |
|---|---|
| AGCGACTAGGCCATAGAGC | (SEQ ID NO: 35) |
| CATCTACCTGAGGCTTTGC | (SEQ ID NO: 36) |
| AGCGACUAGGCCAUAGAGC | (SEQ ID NO: 37) |
| CAUCUACCUGAGGCUUUGC | (SEQ ID NO: 38) |

Additional siRNA sequences of the invention include SEQ ID NO: 18 and 19 and their complementary sequences, as well as the following:

| | |
|---|---|
| AAGACGGCGCAGGCGGCAGCGTT | (SEQ ID NO: 45) |
| AAGAGGCCGGCGGGAUCGUCGTT | (SEQ ID NO: 46) |
| AUAGUAGACUCCAGCCUUGGCTT | (SEQ ID NO: 47) |
| AUUCCGACCUCGGUGAAGGGTT | (SEQ ID NO: 48) |
| CGCUGCCGCCUGCGCCGUCUUTT | (SEQ ID NO: 49) |
| CGACGAUCCCGCCGGCCUCUUTT | (SEQ ID NO: 50) |
| GCCAAGGCUGGAGUCUACUAUTT | (SEQ ID NO: 51) |
| CCCUUCACCGAGGUCGGAAUTT | (SEQ ID NO: 52) |
| GCUCUAUGGCCUAGUCGCUTT; | (SEQ ID NO: 53) |
| GCAAAGCCUCAGGUAGAUGTT | (SEQ ID NO: 54) |
| AGCGACUAGGCCAUAGAGCTT | (SEQ ID NO: 55) |
| CAUCUACCUGAGGCUUUGCTT. | (SEQ ID NO: 56) |

An siRNA of the invention may also be chemically modified to increase its stability under physiological conditions such as in serum, in the circulation, or in a mammalian cell. Chemically-modified siRNAs of the invention include those that are cholesterol-conjugated, lipid encapsulated and antibody-linked siRNAs. Small interfering RNAs of the invention may also be conjugated to other hydrophobic lipid molecules such as high density lipoprotein to form lipophilic conjugates, or they may have a partial phosphorothioate backbone and 2′-O-methyl sugar modifications on the sense or antisense strands. Lipophilic conjugates of siRNAs include those conjugated to saturated, alkyl chains such as stearoyl (C18) and docosanyl (C22) as well as those conjugated to a hybrid of lithocholic acid and oleylamine (lithocholic-oleyl, C43).

4-1BBL-Specific Ribozyme

A 4-1BBL blocking agent may also be a ribozyme. A ribozyme is an RNA molecule with catalytic activity that is capable of cleaving a single-stranded nucleic acid such as an mRNA that has a homologous region. See, for example, Cech, Science 236: 1532-1539 (1987); Cech, Ann. Rev. Biochem. 59:543-568 (1990); Cech, Curr. Opin. Struct. Biol. 2: 605-609 (1992); Couture and Stinchcomb, Trends Genet. 12: 510-515 (1996). A ribozyme may be used to catalytically cleave a 4-1BBL mRNA transcript and thereby inhibit translation of the mRNA. See, for example, Haseloff et al., U.S. Pat. No. 5,641,673. A ribozyme having specificity for a 4-1BBL nucleic acid may be designed based on the nucleotide sequence of SEQ ID NO: 3 and 4. Methods of designing and constructing a ribozyme that can cleave an RNA molecule in trans in a highly sequence specific manner have been developed and described in the art. See, for example, Haseloff et al., Nature 334:585-591 (1988). A ribozyme may be targeted to a specific RNA by engineering a discrete “hybridization” region into the ribozyme. The hybridization region contains a sequence complementary to the target RNA that enables the ribozyme to specifically hybridize with the target. See, for example, Gerlach et al., EP 321,201. The target sequence may be a segment of about 10, 12, 15, 20, or 50 contiguous nucleotides selected from the nucleotide sequence of SEQ ID NO: 3 or 4. Longer complementary sequences may be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target. Alternatively, an mRNA encoding a 4-1BBL may be used to select a catalytic RNA having a specific ribonuclease activity from a pool of RNA molecules. See, for example, Bartel & Szostak, Science 261:1411-1418 (1993).

Other 4-1BBL Blocking Agents

4-1BBL blocking agents also include inhibitors of other molecules that are involved in 4-1BBL expression or activity, including oligonucleotides that reduce expression of these molecules or inhibitors of the activity of the molecules themselves. Molecules that are involved in 4-1BBL expression or activity include, for example, NF-κB; CREB; C/EBP; the TLRs including TLR2, TLR3, TLR4, TLR7, TLR8, and TLR9; MyD88; TRIF; p38; Jnk; Mek; and TRAF6. Oligonucleotides that reduce expression of these molecules are similar to oligonucleotides such as those described above for the oligonucleotide-based 4-1BBL blocking agents. These would include siRNA (e.g. SEQ ID NO: 18 and 19), an antisense nucleic acid or a ribozyme directed against the selected molecule that is involved in 4-1BBL expression or activity. Activity inhibitors include, for example, the NF-κB inhibitor sulfasalazine, the proteasome inhibitor MG-132, the p38 inhibitor SB203580, the Jnk inhibitor SP600125, and the Mek inhibitor PD98059.

Reducing Inflammation

4-1BBL blocking agents can be employed to prevent or treat inflammatory disease conditions. 4-1BBL blocking agents will reduce inflammation by reducing the over-production of an inflammatory cytokine such as, for example, TNF or IL-6. Thus, in one embodiment, the invention provides a method for reducing production of an inflammatory cytokine in a mammal. The mammal can be one suffering from an inflammatory condition or a mammal at risk for such a condition.

Inflammatory conditions include, without limitation, rheumatoid arthritis, juvenile rheumatoid arthritis, psoriatic arthritis, psoriasis, ankylosing spondylitis, Crohn's disease, irritable bowel syndrome, systemic-onset juvenile chronic arthritis, and osteoporosis. A mammal suffering from an inflammatory condition may be identified based on known symptoms of the condition. For example, a mammal suffering from Crohn's disease or rheumatoid arthritis may exhibit an increase in the level or duration of production of pro-inflammatory cytokines such as, for example, IL-1, IL-6, MCP-1 and TNF compared to an unafflicted mammal of the same species. The increase can be any amount such as, for example, 5%, 10%, 15%, 20% or more than 20%. Other symptoms of an inflammatory condition include, without limitation, pathologic inflammation and joint destruction. A mammal at risk for an inflammatory condition may be identified based on genetic aberration as discussed in, for example, Vyse & Todd, Cell 85:311-18 (1996); Kinne et al. Arthritis Research 3:319-330 (2001) and Rioux & Abbas, Nature 435:584-9 (2005)).

4-1BBL blocking agents may be administered in numerous ways. Examples of routes of administration include parenteral, intravenous, intradermal, subcutaneous, oral, by inhalation, transdermal (topical), transmucosal, and rectal administration. Systemic administration may be by transmucosal or transdermal means. A polypeptide-based blocking agent, for example, may be administered by injection under the skin as needed or as determined by the physician. These may also be injected by IV infusion over several hours. A oligonucleotide-based 4-1BBL blocking agent such as an antisense or siRNA may be inserted into a vector and used as gene therapy vector. A gene therapy vector may be delivered to a subject by intravenous injection, local administration or by stereotactic injection. See, for example, U.S. Pat. No. 5,328,470 and Chen et al., PNAS 91:3054 (1994). Thus, a 4-1BBL blocking agent may be administered to a subject by direct injection at a tissue site or generated in situ. Alternatively, it may be modified to target selected cells and then administered systemically. For example, antisense molecules may be modified such that they bind to receptors or antigens expressed on a selected cell surface. Other small molecule type blocking agents may be administered orally.

4-1BBL blocking agents may be used alone or in combination with a second medicament, e.g. a known anti-inflammatory medication such as, for example, Adalimumab (Humira®), Efalizumab (Raptiva®), Etanercept (Enbrel), Infliximab (Remicade®), methotrexate (Rheumatrex), and Omalizumab (Xolair®).

The dosage to be administered to a mammal may be any amount appropriate to reduce 4-1BBL expression or activity. The dosage may be an effective dose or an appropriate fraction thereof. This will depend on individual patient parameters including age, physical condition, size, weight, the condition being treated, the severity of the condition, and any concurrent treatment. Factors that determine appropriate dosages are well known to those of ordinary skill in the art and may be addressed with routine experimentation. For example, determination of the physicochemical, toxicological and pharmacokinetic properties may be made using standard chemical and biological assays and through the use of mathematical modeling techniques known in the chemical, pharmacological and toxicological arts. The therapeutic utility and dosing regimen may be extrapolated from the results of such techniques and through the use of appropriate pharmacokinetic and/or pharmacodynamic models. The precise amount to be administered to a patient will be the responsibility of the attendant physician. However, 4-1BBL blocking agents may be administered orally or by injection at a dose of from about 0.05 to about 2000 mg/kg weight of the mammal, preferably from about 1 to about 200 mg/kg weight of the mammal. An oligonucleotide-based blocking agent may be administered (e.g., orally or by injection) at a dose of from 0.05 to 500 mg/kg weight of the mammal, preferably 0.5 to 50 mg/kg weight of the mammal. The dose range for adult humans is generally from 4 to 40,000 mg/day and preferably 40 to 4,000 mg/day. As certain agents of the invention are long acting, it may be advantageous to administer an initial dose of 80 to 4,000 mg the first day then a lower dose of 20 to 1,000 mg on subsequent days. A patient may also insist upon a lower dose or tolerable dose for medical reasons, psychological reasons or for virtually any other reasons. The effective amount of the second medicament formulated as a component of a pharmaceutical combination will follow the recommendations of the second medicament manufacturer, the judgment of the attending physician and will be guided by the protocols and administrative factors for amounts and dosing as indicated in the PHYSICIAN'S DESK REFERENCE.

The effectiveness of the method of treatment can be assessed by monitoring the patient for improvements in known signs or symptoms of a disorder. For example, amelioration of inflammation can be detected by monitoring levels and duration of pro-inflammatory cytokine production, e.g. IL-1, IL-6, TNF or MCP-1 production. Any decrease in level or duration of pro-inflammatory cytokine production, e.g. a decrease of 5%, 10%, 15%, 20% or more than 20% indicates an improvement in the patient.

Pharmaceutical Compositions of 4-1 Blocking Agents

A 4-1BBL blocking agent may be incorporated into a pharmaceutical composition that is suitable for administration to a mammal (herein a pharmaceutical composition of the invention). A pharmaceutical composition of the invention typically comprises the active agent and a pharmaceutically acceptable carrier. As used herein, the phrase “pharmaceutically acceptable carrier” means any and all solvents, dispersion media, coatings, antibacterial and antifungal agents, isotonic and absorption delaying agents, and the like known in the art to be compatible with pharmaceutical administration to a mammal. Except insofar as any conventional media or agent is incompatible with the active compound, use thereof in the pharmaceutical composition of the invention is contemplated. In addition, supplementary active compounds may also be included into the pharmaceutical compositions. For example, the pharmaceutical composition of the invention may also include a second anti-inflammatory medicament as described above.

A therapeutically effective amount of the blocking agent can be used in the compositions and methods of the invention. Such a therapeutically effective amount of blocking agent is generally sufficient to reduce 4-1BBL expression or activity in a cell or tissue of interest, or in a mammal. In some embodiments the therapeutically effective amount of the blocking agent is an amount sufficient to reduce inflammation. Examples of therapeutically effective amounts of blocking agents are the dosages described in the foregoing section. Thus, 4-1BBL blocking agents may be administered at a dose of from about 0.05 to about 2000 mg/kg weight of the mammal, preferably from about 1 to about 200 mg/kg weight of the mammal. An oligonucleotide-based blocking agent may be administered orally or by injection at a dose of from 0.05 to 500 mg/kg weight of the mammal, preferably 0.5 to 50 mg/kg weight of the mammal, for example, 1, 3, 5 mg/kg. The dose range for adult humans is generally from 4 to 40,000 mg/day and preferably 40 to 4,000 mg/day. As certain agents of the invention are long acting, it may be advantageous to administer an initial dose of 80 to 4,000 mg the first day then a lower dose of 20 to 1,000 mg on subsequent days.

A pharmaceutical composition of the invention is formulated to be compatible with the intended route of administration. Solutions or suspensions used for parenteral, intradermal or subcutaneous application may include (1) a sterile diluent such as water for injection, saline solution, fixed oils, polyethylene glycols, glycerine, propylene glycol or other synthetic solvents; (2) antibacterial agents such as benzyl alcohol or methyl parabens; (3) antioxidants such as ascorbic acid or sodium bisulfite; (4) chelating agents such as ethylenediaminetetraacetic acid; (5) buffers such as acetates, citrates or phosphates and agents for the adjustment of tonicity such as sodium chloride or dextrose. pH may be adjusted with acids or bases, such as hydrochloric acid or sodium hydroxide. The parenteral preparation may be enclosed in ampoules, disposable syringes or multiple dose vials.

Pharmaceutical compositions may also be formulated and administered based on the nature of the 4-1BBL blocking agent. Oligonucleotide-type 4-1BBL blocking agents such as siRNA maybe delivered using vector-based delivery systems such as those described in Li & Rossi, Methods Mol. Biol. 309:261-72 (2005), using high-pressure intravenous injections of siRNA and chemically-modified siRNAs including cholesterol-conjugated, lipid encapsulated and antibody-linked as described in Song et al., Nat. Med. 9:347-51 (2003); Soutschek et al., Nature 432:173-178 (2004); Morrissey et al., Nat. Biotechnol. 23:1002-1007 (2005); Song et al., Nat. Biotechnol. 23:709-717 (2005); and Wolfrum et al., Nat. Biotechnol. 25:1149-1157 (2007). Oligonucleotide-type 4-1BBL blocking agents such as siRNA and ribozymes may also be delivered using cationic liposome such as those described by Yano et al., In Vivo Antitumor Activity of a New Cationic Liposome siRNA Complex, in NON-VIRAL GENE THERAPY, Springer Tokyo, 2005.

Pharmaceutical compositions suitable for injection include sterile aqueous solutions or dispersions and sterile powders for extemporaneous preparation of sterile injectable solutions or dispersions. For intravenous administration, suitable carriers include physiological saline, bacteriostatic water, or phosphate buffered saline. Compositions must be sterile and be stable under the conditions of manufacture and storage and must be preserved against contamination by microorganisms such as bacteria and fungi. The carrier may be a solvent or dispersion medium containing, for example, water, ethanol, polyol (e.g. glycerol, propylene glycol and liquid polyethylene glycol), and suitable mixtures thereof. The proper fluidity may be achieved, for example, by using a coating such as lecithin, by maintaining the required particle size in the case of dispersion and by using surfactants. Prevention of the action of microorganisms may be achieved using various antibacterial and antifungal agents such as, for example, parabens, chlorobutanol, phenol, ascorbic acid, and thimerosal. Other ingredients such as an isotonic agent or an agent that delays absorption (e.g. aluminum monostearate and gelatin) may be included.

Sterile injectable solutions may be prepared by incorporating the active agent in the required amount in an appropriate solvent with one or a combination of ingredients discussed above, as required, followed by filtered sterilization. Dispersions may be prepared by incorporating the active compound into a sterile vehicle that contains a basic dispersion medium and other required ingredients discussed above. In the case of sterile powders for the preparation of injectable solutions, the preferred methods of preparation include vacuum drying and freeze-drying which yield a powder of the active ingredient and any additional desired ingredient from a previously sterile-filtered solution.

Oral compositions may include an inert diluent or an edible carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier. Pharmaceutically compatible binding agents and/or adjuvant materials may be included as part of the composition. The tablets, pills, capsules, troches and the like may contain any of the following ingredients or compound of a similar nature: a binder such as microcrystalline cellulose, gum tragacanth or gelatin; an excipient such as starch or lactose; a disintegrating agent such as alginic acid or corn starch; a lubricant such as magnesium stearate; a glidant such as colloidal silicon dioxide; a sweetening agent such as sucrose or saccharin; or a flavoring agent such as peppermint, methyl salicylate or orange flavoring.

For administration by inhalation, the composition may be delivered in the form of an aerosol spray from a pressurized container or dispenser which contains a suitable propellant, for example, a gas such as carbon dioxide or a nebulizer.

For transmucosal or transdermal administration, penetrants known in the art to be appropriate to the barrier to be permeated may be used. These include detergents, bile salts and fusidic acid derivatives for transmucosal administrations, which may be accomplished using nasal sprays, for example. For transdermal administration, the active compounds are formulated into ointments, salves, gels or creams as generally known in the art.

In one embodiment, the agents of the invention may be prepared with carriers that will protect the agent against rapid elimination from the body, such as a controlled release formulation such as implants and microencapsulated delivery systems. Biodegradable biocompatible polymers may be used. These include ethylene vinyl acetate, polyanhydrides, polyglycolic acid, collagen, polyorthoesters, and polylactic acid. Methods for preparation of such formulations will be apparent to those skilled in the art. Liposomal suspensions, including those targeted to infected cells with monoclonal antibodies to viral antigens may also be used as pharmaceutically acceptable carriers. These may be prepared using methods known in the art.

Oral or parenteral compositions may be formulated in dosage unit form for ease of administration and uniformity of dosage. The phrase “dosage unit form” refers to physically discrete units suited as unitary dosages for the subject to be treated, each unit containing a predetermined quantity of active compound calculated to produce the desired therapeutic effect in association with the required pharmaceutical carrier. The specification for the dosage unit forms is dependent on the unique characteristics of the active compound and the particular therapeutic effect to be achieved.

The pharmaceutical preparation of the gene therapy vector may include the to gene therapy vector in an acceptable diluent or may comprise a slow release matrix in which the gene delivery vehicle is imbedded.

A pharmaceutical composition of the invention may be included in a container, pack or dispenser together with instructions for administration. Such articles of manufacture will include instructions for use of the 4-1BBL blocking agent for the treatment of sustained inflammation. In addition, such articles of manufacture may include instructions for use of a second medicament for the treatment of inflammation.

Screening Assays

The invention also encompasses methods of screening for novel 4-1BBL blocking agents. Novel 4-1BBL blocking agents may be identified based on its ability to interfere with 4-1BBL oligomerization, 4-1BBL interaction with other signaling molecules such as TRAF6 or TLRs, or 4-1BBL translocation to the cell membrane.

A 4-1BBL blocking agent that can prevent 4-1BBL oligomerization can be identified in a cell based assay in the presence and absence of a candidate agent. A candidate agent, the presence of which mediates the binding of two 4-1BBL, is a 4-1BBL blocking agent in that it would prevent oligomerization of 4-1BBL, i.e. the formation of an aggregate of three or more 4-1BBL. In general, the binding of two 4-1BBL can be detected using standard methods including, without limitation, the yeast two hybrid systems (see Fields & Song, Nature 340:245-46 (1989) and the protein fragment complementation assay (see http://www.abrforg/JBT/Articles/JBT0012/jbt0012.html (last visited Sep. 8, 2006) such as the β-lactamase complementation assay. In the yeast two-hybrid system, two 4-1BBL fusion proteins can be expressed in a yeast cell. One fusion protein consist of 4-1BBL fused to a DNA-binding domain, and a second fusion protein consist of 4-1BBL fused to a transcription activation domain. The binding of 4-1BBL leads to activation of a reporter gene that allows the yeast to grow on a defined medium. In the β-lactamase complementation assay, two β-lactamase enzyme fragments, Bla(a) and Bla(b), which consist of amino acids 26-196 and 198-290, respectively, can be fused with 4-1BBL by a (Gly₄Ser)₃ linker. Cells transfected with a pair of 4-1BBL-Bla(a) and 4-1BBL-Bla(b) are loaded with CCF2/AM. CCF2/AM diffuses across the cell membrane, and the cytoplasmic esterases hydrolyze its ester functionalities, releasing the β-lactamase substrate CCF2. Excitation of the coumarin donor in CCF2 at 409 nm leads to FRET to the fluorescein acceptor generating emission of green fluorescence at 520 nm. The binding of two 4-1BBL brings two β-lactamase fragments into proximity resulting in a complete form of β-lactamase. This β-lactamase further hydrolyzes CCF2 and separates donor and acceptor leading to FRET disruption. As a result, the isolated coumarin donor emits blue fluorescence at 447 nm. This can be observed under the microscope or measure by flow cytometer. The binding of two

A 4-1BBL blocking agent that can inhibit or reduce 4-1BBL interaction with signaling molecules can be identified using cell-based methods. Examples of such assays are described herein. A cell-based method for identifying a 4-1BBL blocking agent that can inhibit or reduce 4-1BBL interaction with signaling molecules, for example, TLR or TRAF6, with (b) a candidate agent, and then determining whether 4-1BBL interacts with the signaling molecule. Alternatively, an agent that inhibits 4-1BBL interaction with TLR or TRAF6 can be identified using a cell based screen similar to that described above for 4-1BBL aggregation. For example, 4-1BBL-yeast DNA binding protein and TRAF6-yeast transcription activator domain, or TRAF6-yeast DNA binding protein and 4-1BBL-yeast transcription activator domain, can be used in a yeast two-hybrid system as described above. Similarly, fusion proteins consisting of 4-1BBL-Bla(a) and TRAF6-Bla(b) or TRAF 6-Bla(a) and 4-1BBL-Bla(b) can be used to assay the interaction of 4-1BBL and TRAF6 as described above.

A 4-1BBL blocking agent that can inhibit or reduce 4-1BBL translocation to the cell surface can be identified by contacting a cell that expresses 4-1BBL with a selected agent and determining whether the 4-1BBL is localized to the cell membrane. Such a cell can be a mammalian cell such as RAW264.7 cell line.

A 4-1BBL blocking agent can also be identified using a cell-based or animal model of inflammation. Examples of cell-based assays are described herein. For example, a cell that expresses an inflammatory cytokine such as TNF, IL-6, IL-1 or MCP-1 in response to the appropriate stimulation, e.g. exposure to LPS, is contacted with a candidate agent. Inflammatory cytokine production by a cell that has been to contacted with the candidate agent is compared with that in a cell that has not been contacted with the candidate agent. For cell-based assays, any cell capable of producing an inflammatory cytokine in response to a stimulation such as LPS that triggers inflammation can be used. These cells include, for example, a RAW264.7 cell, a primary murine macrophage, and a primary human monocyte. Similarly, 4-1BBL blocking agents can be identified using an animal model of inflammation. For example, an mammal can be administered a candidate agent and the production of inflammatory cytokine such as TNF or IL-6, or other observable phenotypes associated with over-sustained inflammation such as increase of body temperature or joint swelling can be determined and compared with that of a similar animal that has not been administered the candidate agent.

The invention will be further described in the following examples, which do not limit the scope of the invention described in the claims.

### EXAMPLES

### Example 1

### Materials and Methods

Mice and reagents. TLR2-, TLR4-, MyD88-, TRIF (LPS2) and both TRIF- and MyD88-deficient mice were as described by Hoebe et al., Nature 424:743-48 (2003); and Kim et al., J. Exp. Med. 197:1441-52 (2003). 4-1BBL-deficient mice were backcrossed eight times to C57BL/6 mice as described by DeBenedette et al., J. Immunol. 163:4833-41 (1999). 4-1BB-deficient mice were backcrossed at least seven times with C57BL/6 mice as described by Vinay, J. Immunol. 173:4218-29 (2004). Sex- and age-matched mice were used in the same experiments.

The preparation and culture of peritoneal macrophages, RAW264.7 and 293T cells are as described by Kim et al., J. Exp. Med. 197:1441-52 (2003). Sex- and age-matched 4-1BBL-deficient and wild-type (WT) mice were injected intraperitoneally with LPS. Blood sera samples were collected or the survival was monitored. The protocols for the use of animals were approved by the Institutional Animal Care and Use Committee at The Scripps Research Institute.

The following reagents were used: Pam₃CysSer(Lys)₄ (EMC microcollections GmbH); LPS from E. coli O111:B4 (List Biological Laboratories); phosphorothioate-stabilized CpG DNA and R-848 (Invivogen); poly I:C (Amersham Biosciences); peptidoglycan (Fluka); IL-1β and TNF (R&D systems); recombinant mouse 4-1BB-Fc and anti-4-1BBL (R&D systems); monoclonal antibodies for 4-1BBL (clone TKS-1, e-Bioscience); anti-Flag were (Sigma); anti-GFP were (Clontech); antibodies against HA, Myc, MyD88, TLR4, and TRAF6 (Santa Cruz Biotechnology); Alexa Fluor 594-conjugated donkey anti-goat IgG (Molecular Probes); anti-AU-1 (Covance); anti-GAPDH (Chemicon); brefeldin A (Biolegend); and sulfasalazine, SB203580, SP600125, and PD98059 (Calbiochem).

Plasmids, recombinant adenoviruses and PCR. Mammalian expression vectors were constructed using either full-length, extracellular domain-deleted (ΔExt), or cytosolic domain-deleted (ΔCyt) human 4-1BBL cDNA, or they were constructed with full-length, pro712his mutant, extracellular domain-deleted (ΔExt), or cytosolic domain-deleted (ΔCyt) human TLR4 cDNA. These cDNAs were fused with either GFP (in pEGFP-C1), Flag (in pcDNA3), or HA (in pcDNA3). Replication-defective adenovirus-5-expressing mouse 4-1BBL (Adv-4-1BBL), or no transgene as a control (Adv-control), were generated using the ‘two plasmid rescue’ method as described for human 4-1BBL in Bukczynski et al., proc. Natl. Acad. Sci. U.S.A. 101:1291-1296 (2004).

Semiquantitative PCR was performed as described by Kim et al., J. Exp. Med. 197:1441-52 (2003). For real-time PCR, 0.5 μg of total RNA were used to prepare cDNA using oligo(dT)₁₂ as a primer. The SYBR green PCR Master Mix kit (Applied Biosystems) was used in the real-time PCR analysis.

Full-length, cytosolic domain, extracellular domain-deleted (ΔExt), or cytosolic domain-deleted (ΔCyt) human 4-1BBL cDNA was cloned into mammalian expression vectors fused with GFP (pEGFP-C1), flag (in pcDNA3), or HA (in pcDNA3). Replication-defective adenovirus-5 expressing mouse 4-1BBL (Adv-4-1BBL) or no transgene as a control (Adv-control), were generated using the same method, as described previously by Bukczynski et al., proc. Natl. Acad. Sci. U.S.A. 101:1291-1296 (2004). Oligonucleotides were cloned into pSuppressor to express siRNA strands targeting mouse 4-1BBL (#1,5′-GCTCTATGGCCTAGTCGCT-3′ (SEQ ID NO: 14); #2,5′-GCAAAGCCTCAGGTAGATG-3′ (SEQ ID NO: 15)), mouse 4-1BB (#1,5′-ACCTGTAGCTTGGGAACATTT-3′ (SEQ ID NO: 16); #2, 5′-AATACAATCCAGTCTGCAAGA-3′ (SEQ ID NO: 17)), or nothing (control). Human IL3R, TLR 2, 3, 4 or 9 cDNA strands were cloned into pcDNA3-flag expression vectors.

Transfection. 293T cells were transfected with the expression vectors using Lipofectamine 2000 following the manufacturer's protocol (Invitrogen), and cell lysates were prepared after 24 hours. For siRNA transfection, mouse macrophage cell line RAW264.7 was transiently transfected with siRNA targeting mouse TRAF6 (#1,5′-GGAUCAUCAAGUACAUUGUTT-3′ (SEQ ID NO: 18); #2,5′-GGGCUACGAUGUGGAGUUUTT-3′ (SEQ ID NO: 19); Pre-designed siRNA from Ambion) or GFP as a control using PolyAmine (Ambion). After 2 days of transfection, cells were incubated with stimulants. Cell lysates or culture supernatants were prepared after 24 hours to analyze the knockdown of TRAF6 by Western blotting or production of TNF-α by ELISA.

Electrophoretic mobility shift, nuclear run-on, and yeast two-hybrid assays. Nuclear extracts were prepared according to the protocol previously described. Radiolabeling of specific oligonucleotides with [γ-³²P] ATP and EMSA were performed as recommended by Promega's protocol. TNF transcription rate was measured by run-on analysis as described in Han et al., Biochim. Biophys. Acta 1090:22-28 (1991). The two-hybrid screening was performed as described using a mixed human fetal brain and spleen cDNA library as described by Han et al., Nature 386:296-299 (1997). A portion of human Tlr4 spanning the intracellular protein domain (amino acids 653-839) was subcloned into pAS2 vector was used as bait. 5×10⁶ transformants were screened.

Generation of 4-1BBL or 4-1BB-knockdown stable cell lines. RAW 264.7 cells were stably transfected with the pSuppressor-4-1BBL, 4-1BB, or empty vector. For the selection of cells, cells were incubated in culture media containing 500 μg/mL of G418 for 2 weeks and then pooled. Cells were maintained in media containing G418.

Flow cytometry. Peritoneal macrophages were suspended in PBS, 5% FCS, 0.01% sodium azide (FACS buffer) on ice. Surface 4-1BBL expression was evaluated using 4-1BBL-specific antibodies (clone TKS-1, eBioscience) conjugated with phycoerythrin. Total 4-1BBL expression was evaluated after the cells were fixed and permeabilized in fixation-permeabilization buffers (Biolegend). The cells were stained with phycoerythrin-conjugated 4-1BBL antibodies in the permeabilization buffer and then washed with FACS buffer. Phycoerythrin-conjugated rat IgG2a, K was used as the isotype control.

Immunohistochemistry and immunoassays. Peritoneal macrophages were seeded on cover glasses and fixed with 4% paraformaldehyde and then permeabilized by incubating in PBS containing 0.5% Triton X-100. After blocking with 2% BSA, cells were incubated with anti-mouse 4-1BBL followed by incubation in Alexa Fluor 594-conjugated donkey anti-goat IgG. Fluorescence images were visualized using AxioVision 3.1 software (Carl Zeiss, Inc.). Supernatants from macrophage culture or mouse blood sera were collected and TNF or IL-6 concentrations were measured by enzyme-linked immunosorbent assay (ELISA) kits (eBioscience) following the manufacturer's protocol. The antibodies used in the immunoprecipitation and immunoblotting procedures are indicated in the figure legends. The experimental procedures are as described in Zhou et al., Mol. Cell. Biol. 26:3824-34 (2006).

Measurement of Cytokines. Supernatants from macrophage culture or mouse blood sera were collected, and TNF-α or IL-6 concentrations were measured by enzyme-linked immunosorbent assay (ELISA) kits (eBioscience) following the manufacturer's protocol.

Statistical analysis. Kaplan-Meier plots were constructed and a log-rank test was used to determine the differences in the survival of mice. The statistical significance of differences in sera TNF was determined by Student's t-test.

### Example 2

### 4-1BBL is a TLR4-Interacting Protein

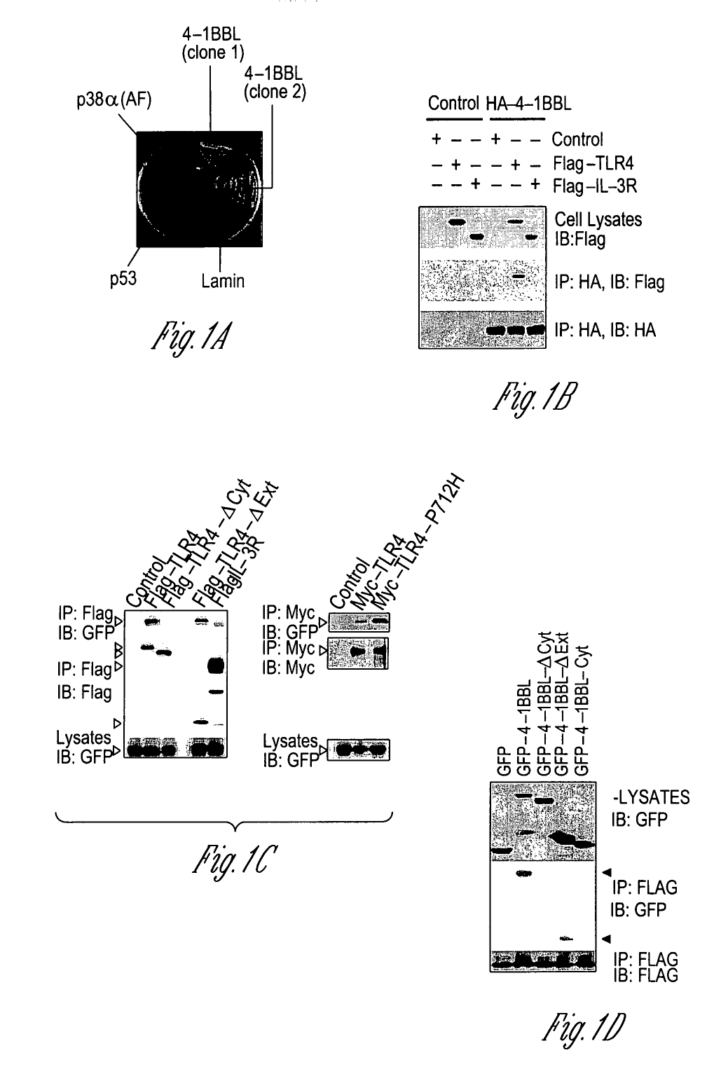

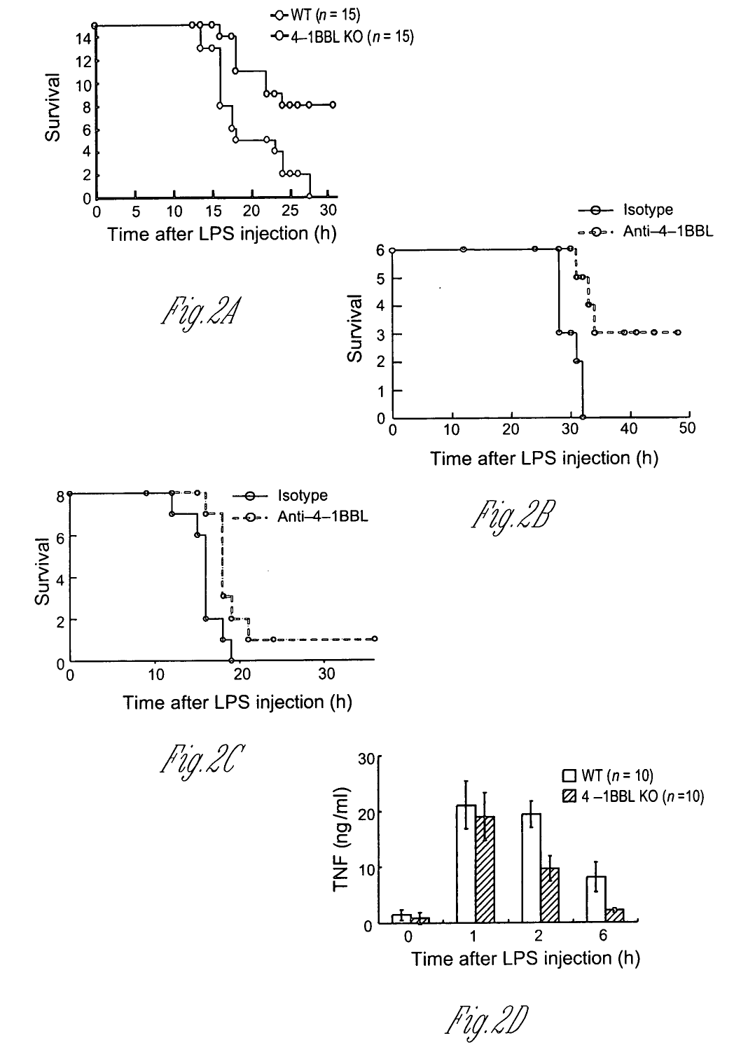

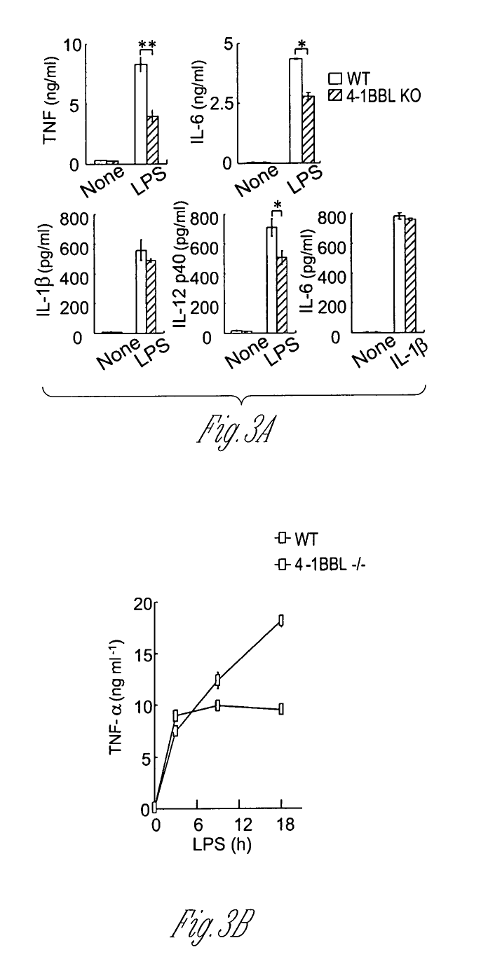

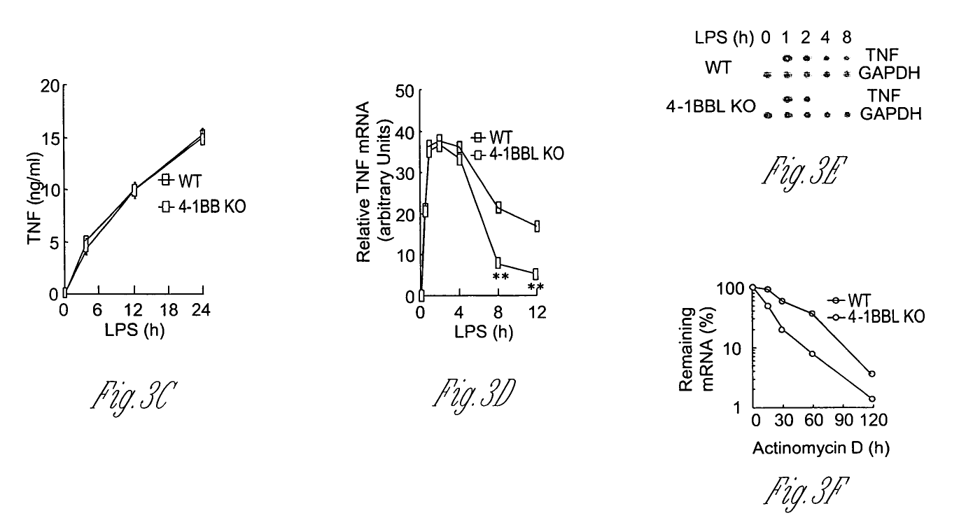

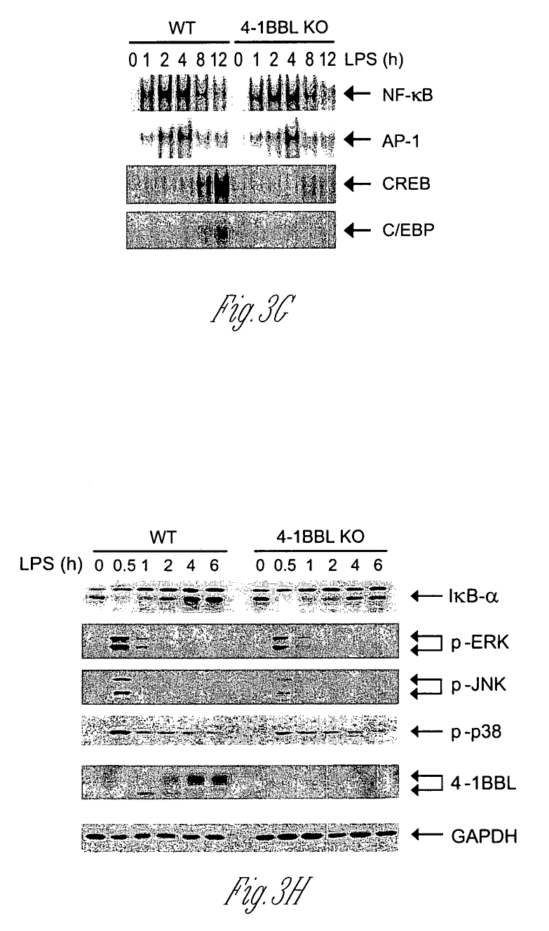

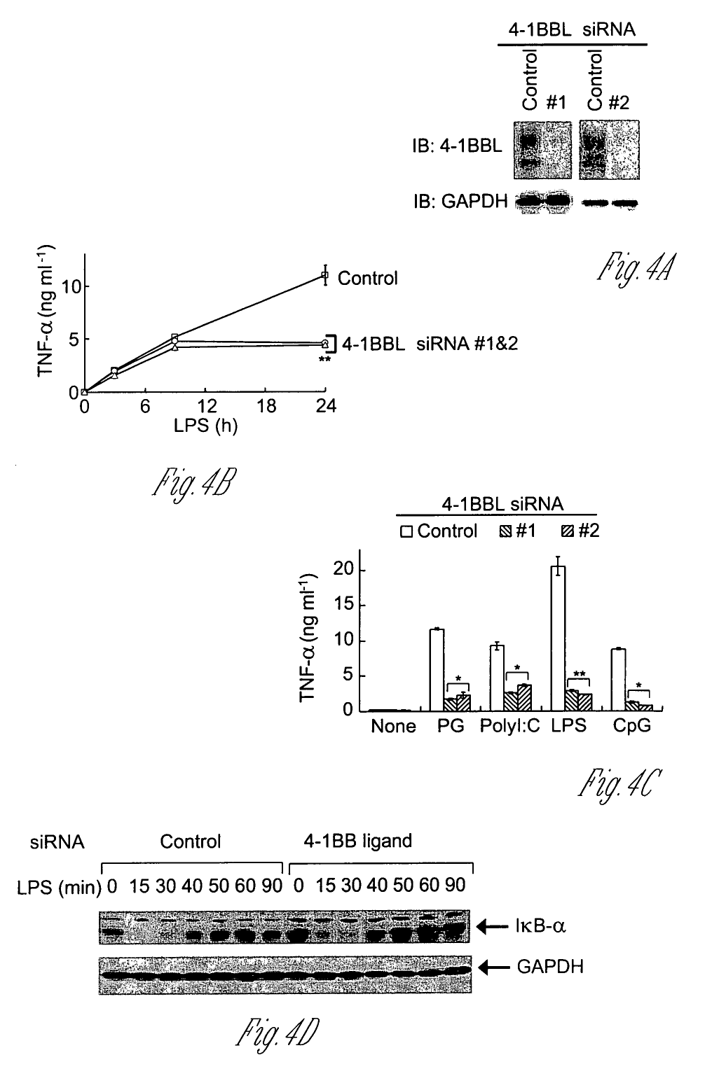

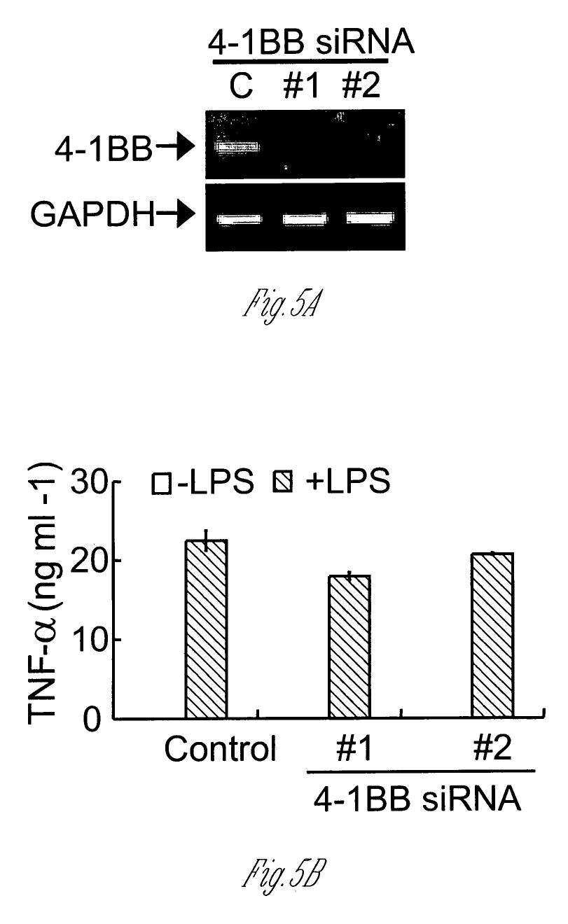

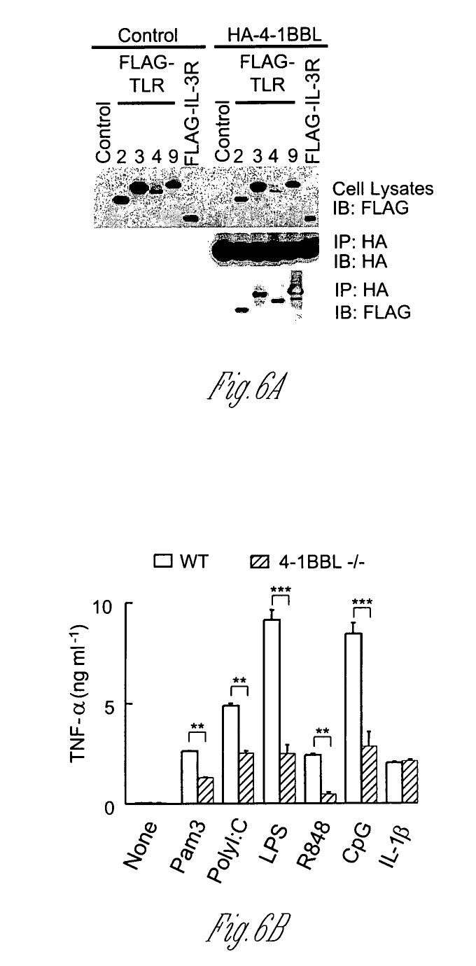

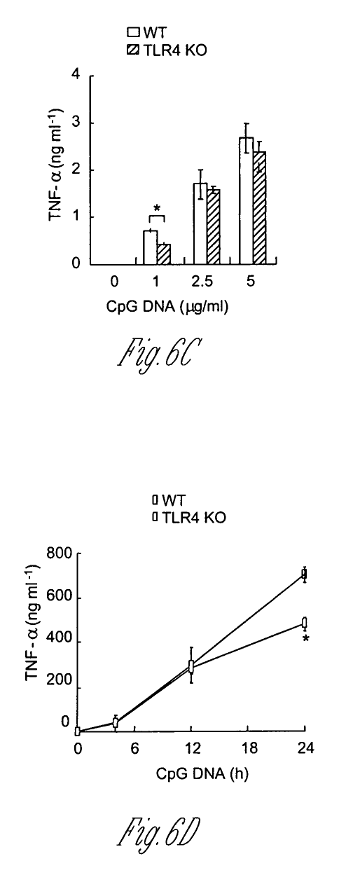

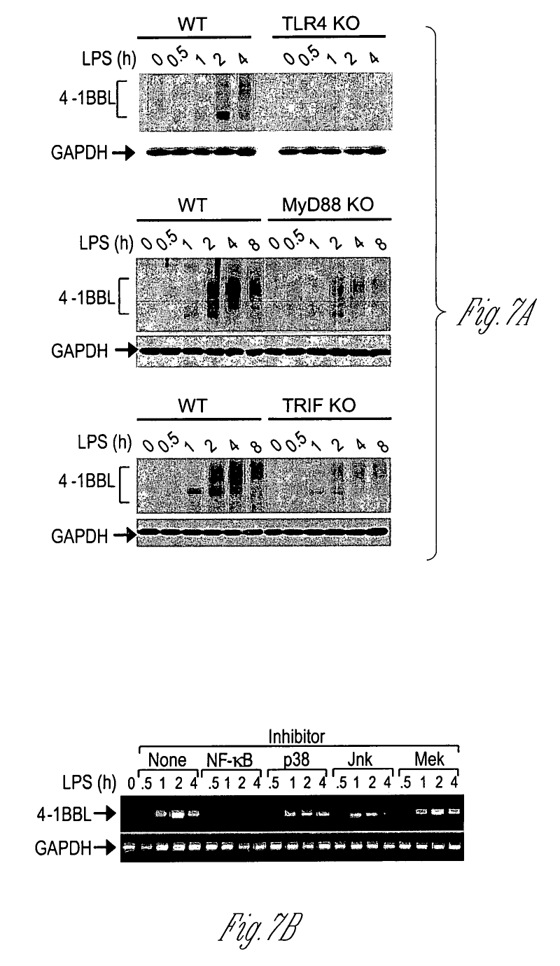

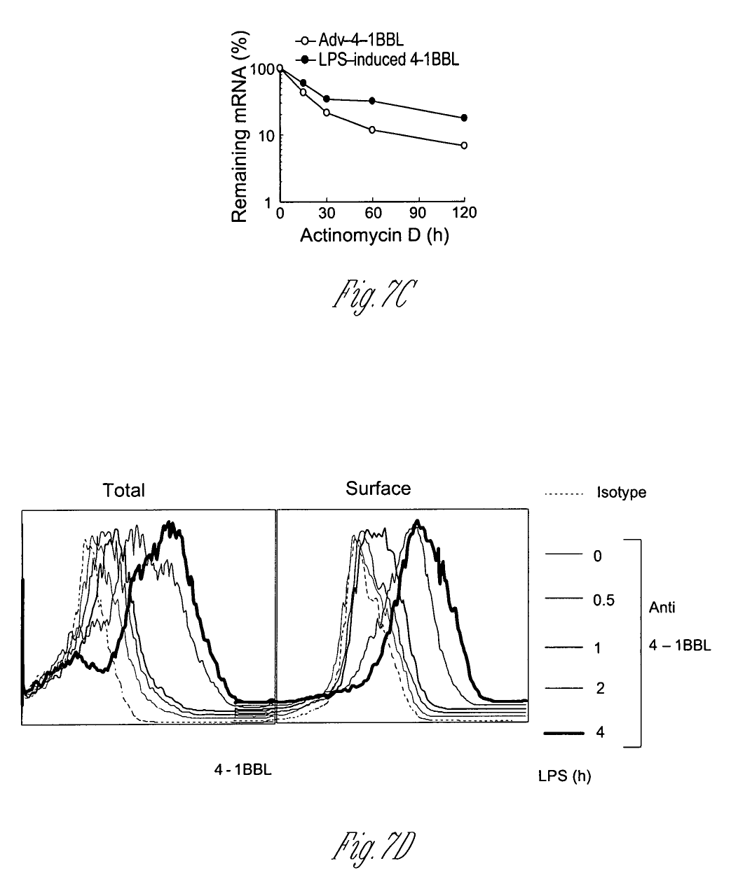

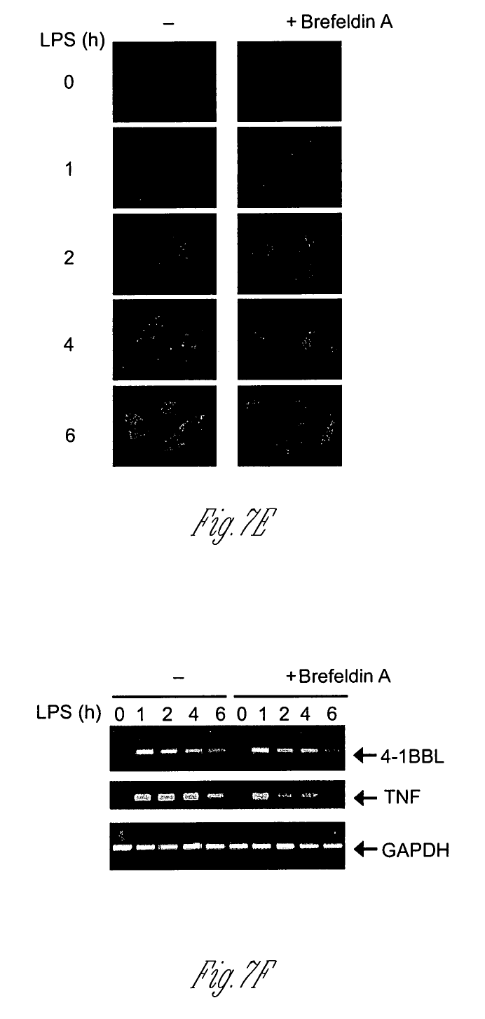

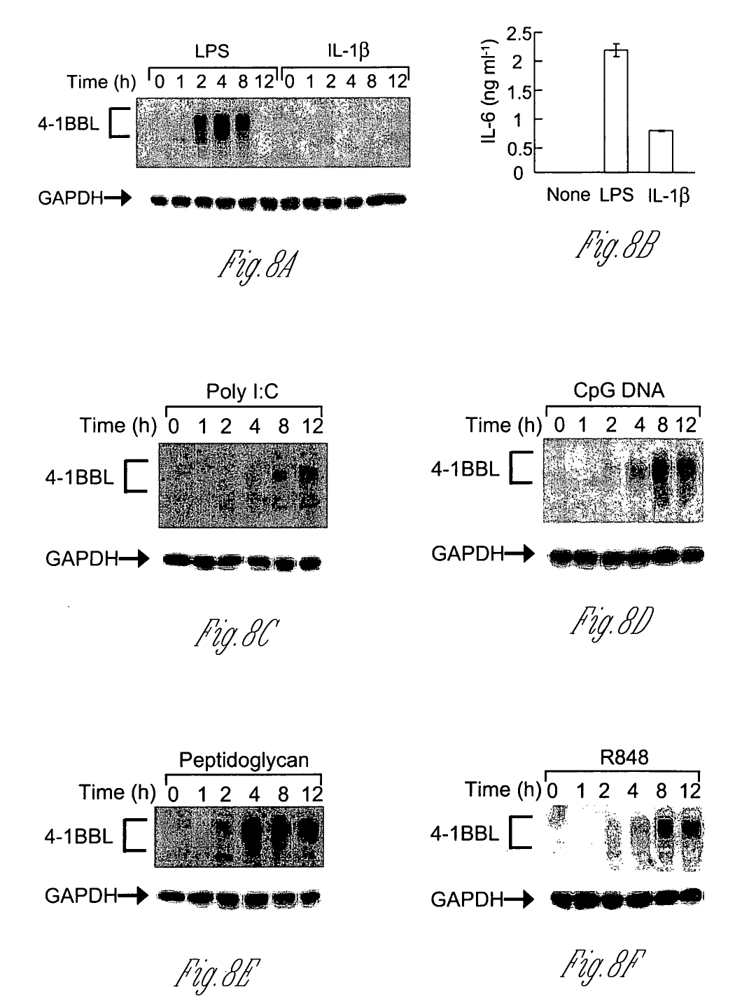

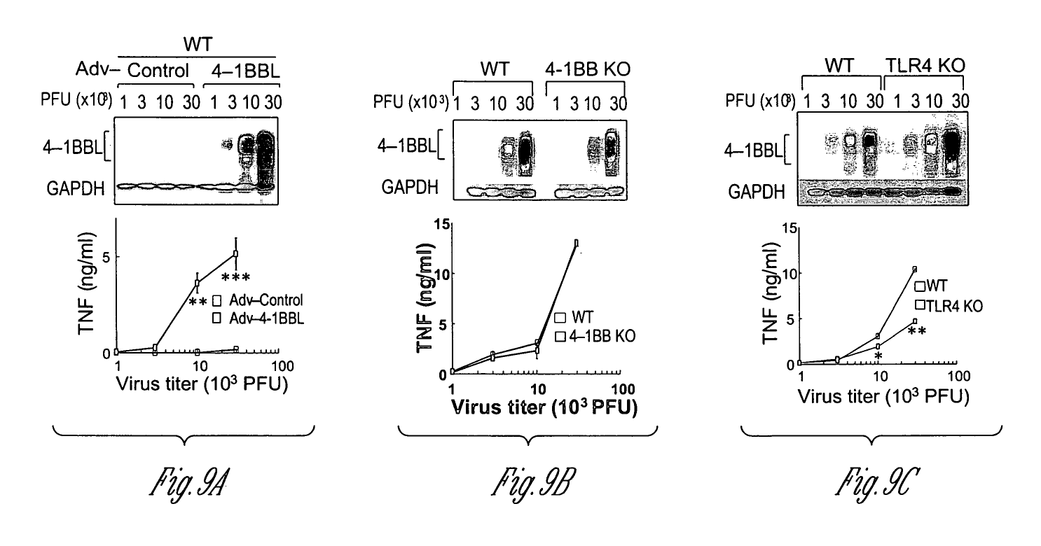

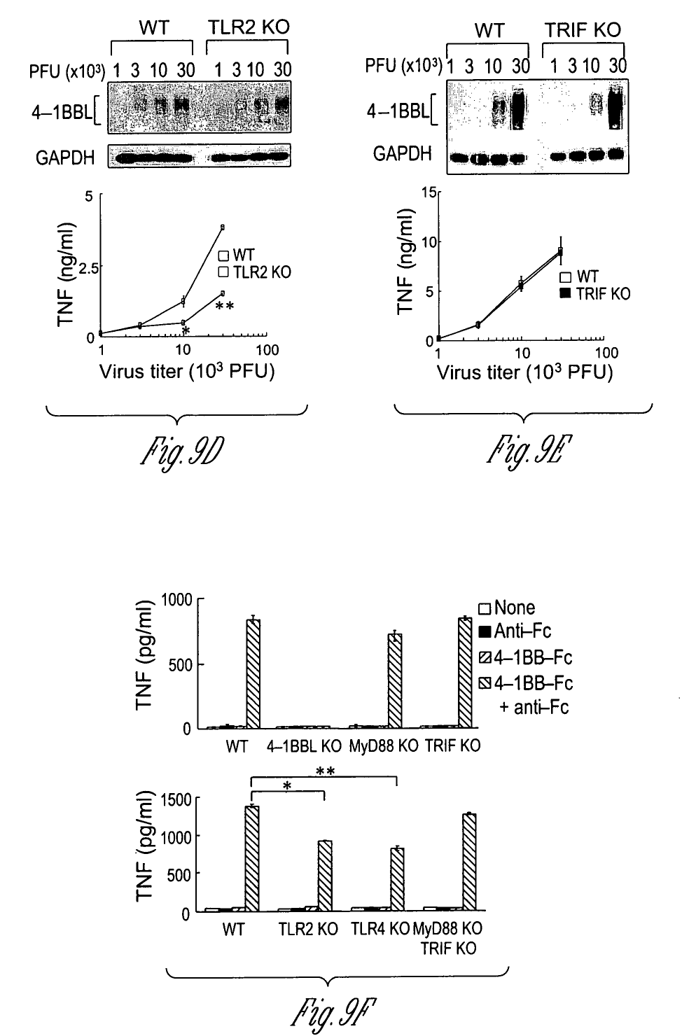

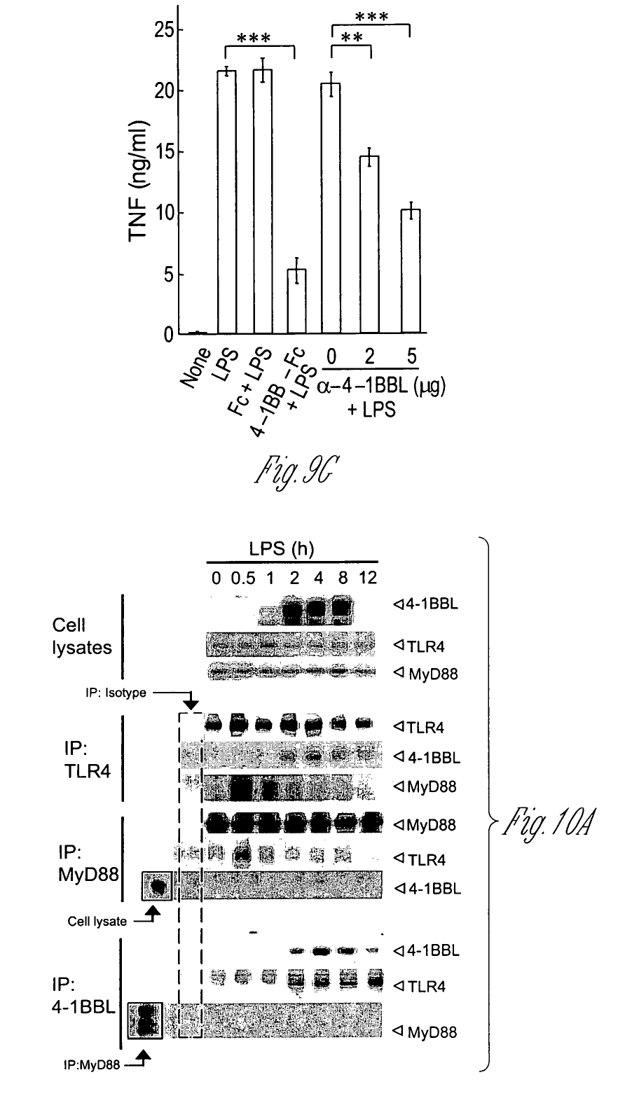

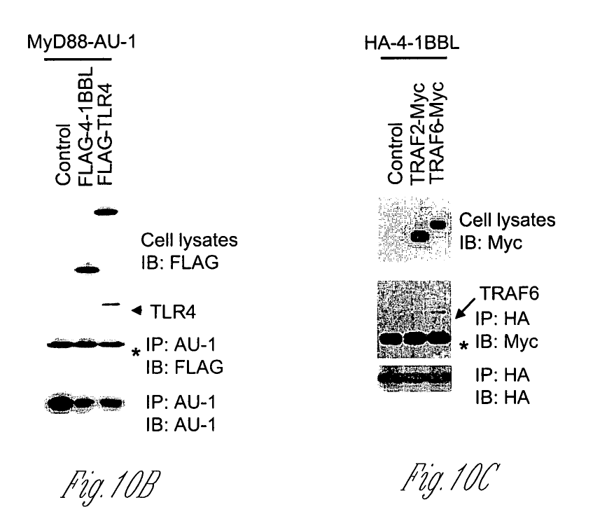

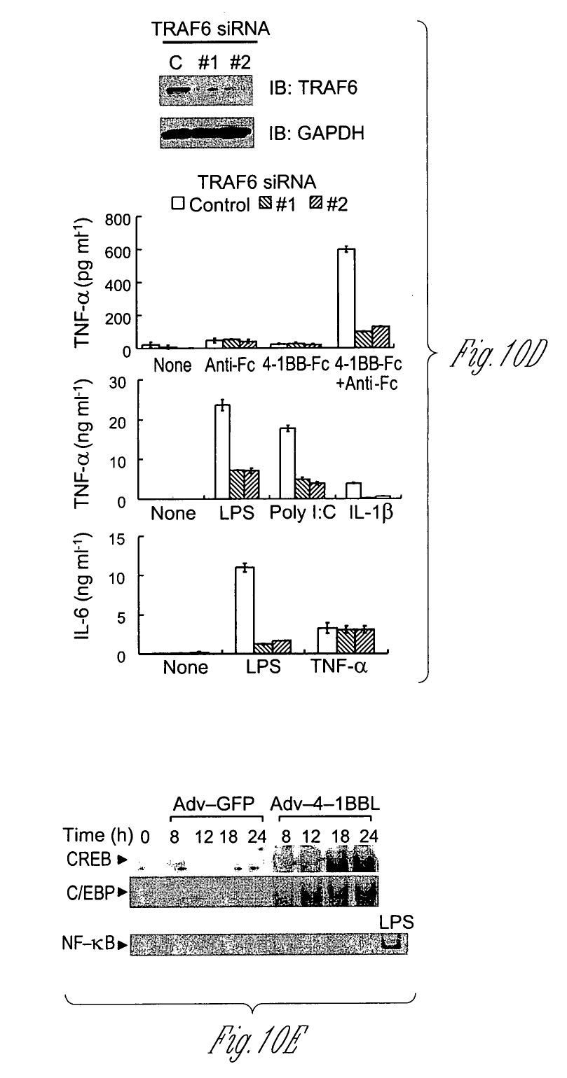

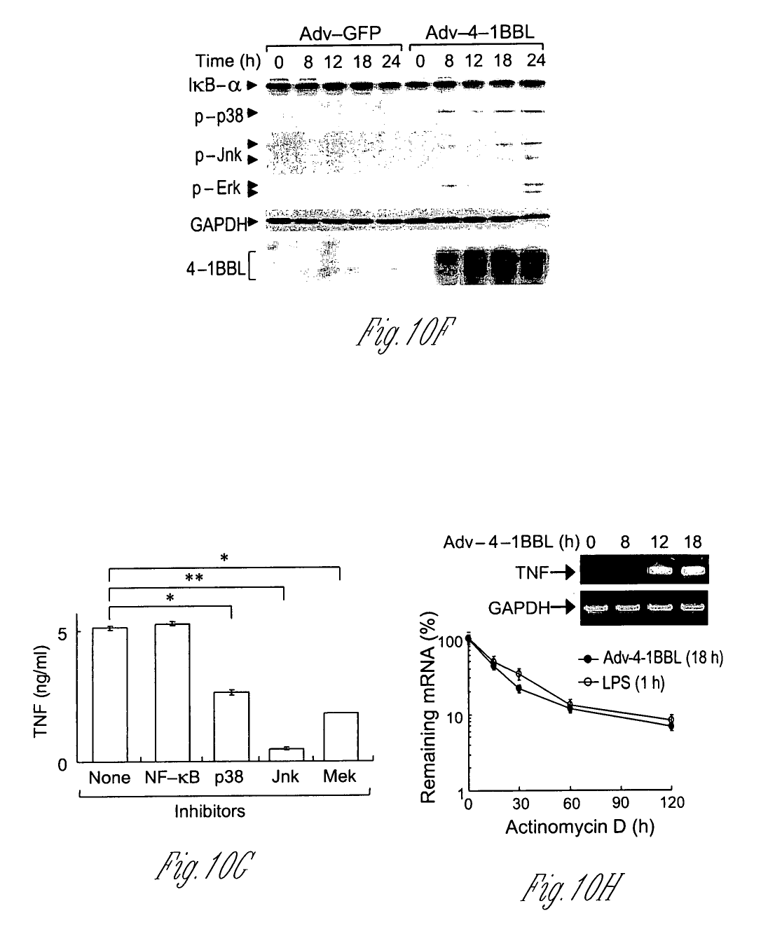

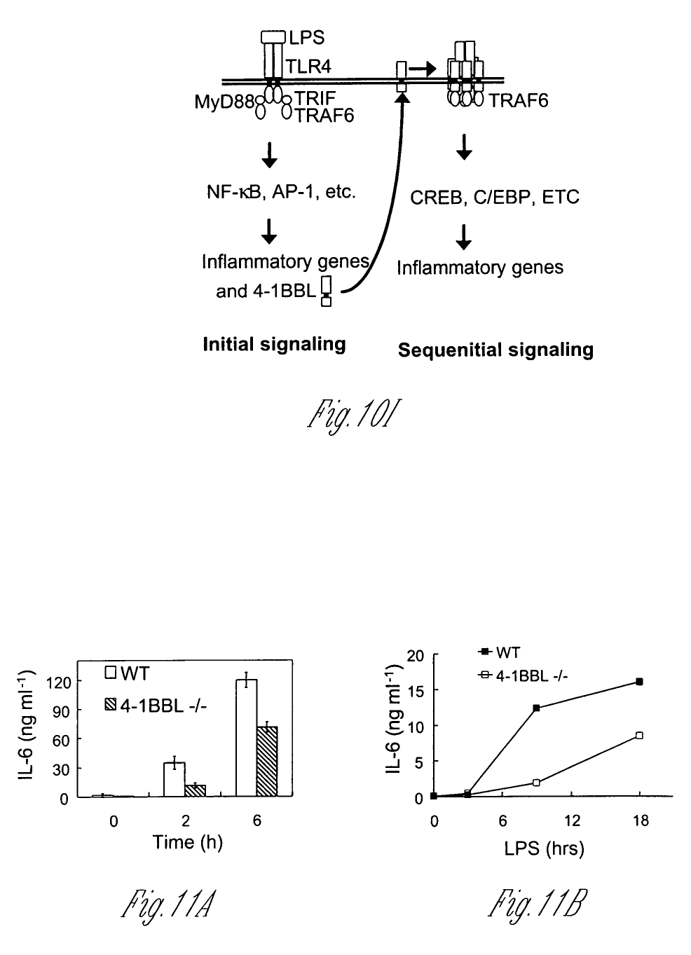

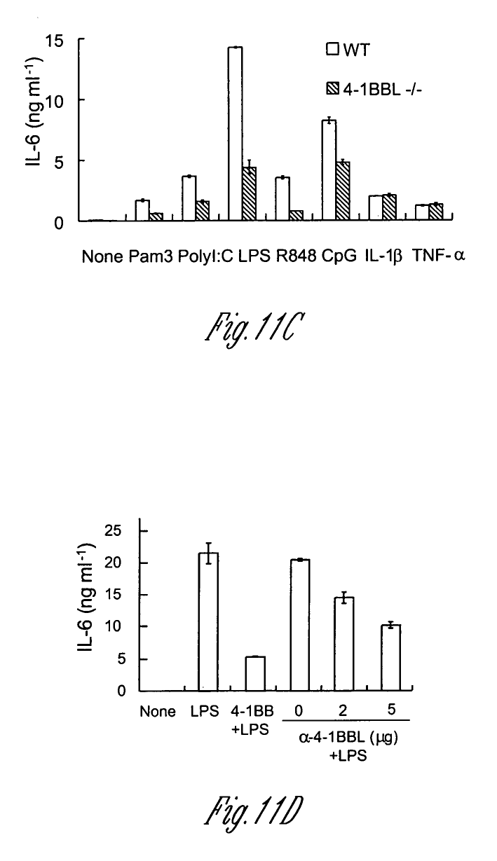

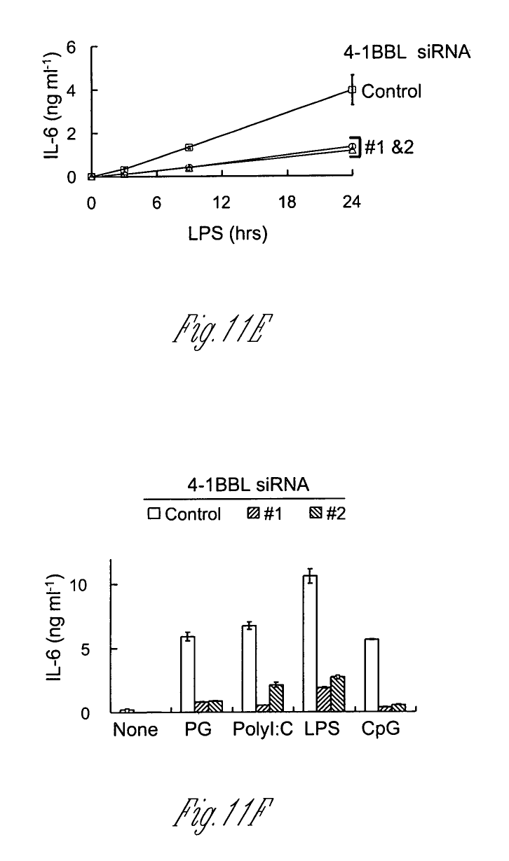

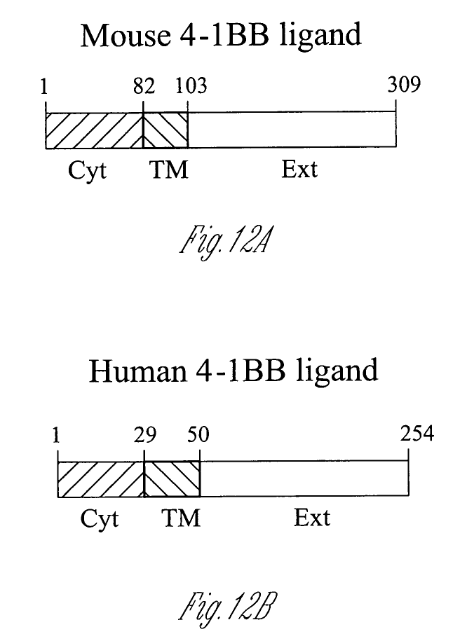

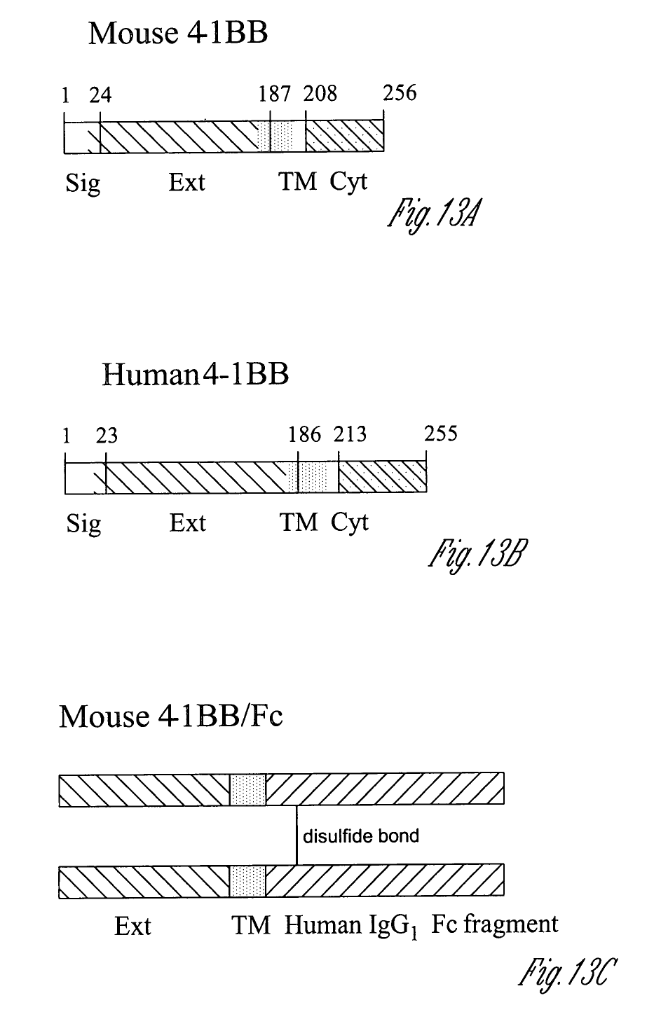

A yeast two-hybrid screen was used to identify proteins that interact with the intracellular domain of TLR4. Yeast cells were transfected with pAS2-TLR4(653-839 aa) together with 4-1BBL (5-254 aa) (Clone 1), 4-1BBL (3-254 aa) (Clone 2), p38((AF), p53, or Lamin in pGAD10 vector. The “bait and prey” interaction was verified by yeast growth on a medium lacking histidine. Two of the seven positive clones were found to be 4-1BBL, a cell surface type II transmembrane protein (see Goodwin et al., Eur. J. Immunol. 23:2631-2641 (1993) and Alderson et al., Eur. J. Immunol. 24:2219-2227 (1994)). 4-1BBL, but not unrelated proteins, interacts with the TLR4 intracellular domain as indicated by the finding that yeast cells containing the TLR4 intracellular domain encoded in the bait vector and 4-1BBL encoded in the prey vector were able to grow in medium lacking histidine due to the interaction-mediated expression of the His3 reporter gene. (FIG. 1A).

To confirm the interaction, HA-4-1BBL was co-expressed with or without flag-TLR4 or flag-IL-3 receptor (IL-3R) in 293 cells, and their association was determined by co-immunoprecipitation. 293T cells were transfected with empty pcDNA3 vector (Control) or the 4-1BBL expression vector in combination with the empty pcDNA3 vector, the flag-TLR4 or flag-IL-3R expression vector. The cells were lysed 24 hours after transfection. ⅔ of the cell lysates were immunoprecipitated with anti-HA antibodies. The lysates and immunoprecipitates were immunoblotted with anti-flag and anti-HA antibodies, and results indicate that TLR4, but not IL-3R, was pulled down by 4-1BBL (FIG. 18).

To characterize the interaction between 4-1BBL and TLR4, the following experiments were conducted.

Immunoassays were conducted using 293T cells transfected with plasmids expressing GFP-4-1BBL and either, Flag-TLR4, Flag-TLR4 without the cytosolic domain (ΔCyt), Flag-TLR4 without the extracellular domain (ΔExt), Flag-IL-3R, Myc-TLR4, or Myc-TLR4-P712H (Lps^{d}). Cell lysates were immunoprecipitated with anti-Flag or anti-Myc, and the cell lysate and immunoprecipitates were immunoblotted with the indicated antibodies. Results in indicate that the TLR4 intracellular domain is involved in its interaction with 4-1BBL, however, mutation (Lps^{d}, Pro712H) in the TLR4 TIR domain had no substantial effect on the interaction between TLR4 and 4-1BBL (FIG. 1C). Because Lps^{d} mutation disrupts the interaction between the TIR domain and MyD88 but does not disturb the structure of the TIR domain (see Xu et al., Nature 408:111-15 (2000)), distinct portions of the TLR4 TIR domain may interact with 4-1BBL and MyD88.

In addition, immunoassays were conducted using 293T cells transfected with the flag-TLR4 expression vector together with vectors expressing GFP, GFP-4-1BBL, GFP-4-1BBL without the cytosolic domain (ΔCyt), GFP-4-1BBL without the extracellular domain (ΔExt), or GFP-4-1BBL just the cytosolic domain (Cyt). The cell lysates were immunoprecipitated with anti-flag antibodies and immunoblotted with anti-flag and anti-GFP antibodies. Results indicate that for 4-1BBL to interact with TLR4, it must contain its cytosolic domain, and it must be associated with the cell membrane, as neither 4-1BBL without the cytosolic domain nor the 4-1BBL cytosolic domain alone co-immunoprecipitated with TLR4 (FIG. 1D).

### Example 3

### Mice Deficient in 4-1BBL are More Resistant to LPS-Induced Death

To determine the in vivo effect of 4-1BBL, 4-1BBL deficient mice were analysed and the results are as follows. Mice lacking 4-1BBL are viable, fertile, and healthy. These mice have normal lymphoid organs, but exhibit defects in CD8⁺T cell memory to viruses. The numbers of peritoneal macrophages in wild-type and 4-1BBL-deficient mice are comparable (data not shown). Intraperitoneal injection of LPS into 4-1BBL-deficient and wild-type mice revealed that 4-1BBL-deficient mice were more resistant than wild-type mice to the lethal effect of LPS (FIG. 2A). Notably, the lethal effect of LPS on wild-type mice was attenuated by administration of anti-4-1BBL (FIG. 2B,C). The in vivo effect of 4-1BBL deletion could be a result of a lack of 4-1BBL-mediated TNF production in macrophages, and/or a lack of activation of 4-1BB by 4-1BBL in cells of other lineages. Interestingly, because they have fewer natural killer (NK) cells and NKT cells, mice lacking 4-1BB are also resistant to death triggered by injection of LPS and galactosamine. However, the LPS resistance in 4-1BBL-deficient and 4-1BB-deficient mice must result from different causes because 4-1BBL-deficient mice contain normal numbers of NK and NKT cells (data not shown).

### Example 4

### 4-1BBL is Involved in Sustained TNF Production

A. In Vivo Studies

To determine whether 4-1BBL is involved in TLR4-mediated host responses, 4-1BBL knockout (KO) and wild type mice were challenged with a 0.5 mg dose of Escherichia coli LPS as follows. 4-1BBL−/− and wild type mice were injected to intraperitoneally with 0.5 mg of LPS, and TNF levels in the sera collected at 2 and 6 hours were measured.

Results indicate that LPS-induced TNF production was much lower in 4-1BBL KO mice in comparison with wild type mice (FIG. 2D). Specifically, LPS induced a quick increase in serum TNF concentrations, which peaked one hour after LPS injection. Serum TNF concentrations in 4-1BBL-deficient mice were about the same as those in wild-type mice at one hour after LPS injection and were lower than those in wild-type mice at later times (FIG. 2D). These data are consistent with the in vitro data discussed below (FIG. 3A) in that 4-1BBL deletion only affected TNF production at later times after LPS treatment; however, the in vivo TNF induction by LPS appeared to be quicker than that in vitro.

B. Studies Using Primary Macrophages

Because macrophages are the primary sources of TNF in LPS-challenged mice (Beutler et al., Nature 316:552-554 (1985)), peritoneal macrophages from 4-1BBL KO and wild type mice were isolated, and TNF production after LPS stimulation were measured.

First, the effects of 4-1BBL on LPS-induced cytokine production in macrophages were determined by measuring cytokines in the medium of 4-1BBL-deficient and wild-type macrophages 24 hours after LPS stimulation. 4-1BBL-deficient macrophages produced less TNF, IL-6, and IL-12 (FIG. 3A) than wild-type macrophages. IL-1β expression was unaffected or only modestly affected by the absence of 4-1BBL (FIG. 3A). 4-1BBL-deficient macrophages produced wild-type quantities of IL-6 after stimulation with IL-1β, suggesting that 4-1BBL is selectively involved in LPS-induced cellular cytokine responses (FIG. 3A).

In addition, TNF production at 6, 12 and 18 hours after LPS induction was also determined. Peritoneal macrophages from 4-1BBL−/− and wild type mice were plated at 2×10⁶ per well in six well dishes and then treated with LPS (100 ng/mL). TNF levels in the media were determined at 6, 12 and 18 hours. Interestingly, TNF production was normal in 4-1BBL KO macrophages during the first three hours of LPS stimulation, but almost completely eliminated after this time point (FIG. 3B) indicating that 4-1BBL was essential to trigger further increases in TNF production, that is during later times after LPS stimulation.

4-1BBL was originally cloned as a ligand of 4-1BB, a T-cell surface receptor that has a co-stimulatory function (see Goodwin et al., Eur. J. Immunol. 23:2631-2641 (1993)). Since 4-1BB is also expressed in macrophages, whether 4-1BB is involved in 4-1BBL-mediated TNF expression in LPS-treated cells was examined. The following experiments were conducted to determine the involvement of 4-1BB in LPS-induced TNF production.

Unexpectedly, the presence of 4-1BB was not required for 4-1BBL to promote sustained LPS-induced TNF production, as the amounts of LPS-induced TNF were similar in wild-type and 4-1BB-deficient macrophages (FIG. 3C). Therefore, 4-1BBL-4-1BB interactions either do not occur in macrophages, or they simply do not play a role in sustaining macrophage TNF production.

C. Studies Using RAW264.7 Macrophages

To determine whether the function of 4-1BBL in the macrophage cell line RAW264.7 is similar to that in primary macrophage cells, siRNA was used to knock down 4-1BBL expression as follows. Briefly, RAW 264.7 cells were stably transfected with pSuppressor containing control siRNA, or 4-1BBL-siRNA#1 or #2. Protein levels of 4-1BBL were measured by immunoblotting. TNF production was measured (1) in control and 4-1BBL knockdown cells treated with LPS for 3, 9 and 24 hours and (2) in control and 4-1BBL knockdown cells treated with peptidoglycan (PG, 10 μg/ml), PolyI:C (25 μg/ml), LPS (100 ng/mL), CpG (5 μg/mL), or untreated culture media (None) for 24 hours. Results show that both siRNAs effectively knocked down 4-1BBL in RAW264.7 cells (FIG. 4A) and inhibited LPS- and other TLR ligand-induced TNF production (FIGS. 4B, C), thus demonstrating that the role of 4-1BBL is the same in RAW264.7 and primary macrophage cells. 4-1BBL has no role in LPS-induced NF-κB activation in RAW264.7 cells since control and 4-1BBL knockdown RAW cells treated with LPS (100 ng/mL) for 15, 30, 40, 50, 60 and 90 minutes showed equal I-κB-α degradation (FIG. 4D).

Next, 4-1BB expression in RAW264.7 cells was knocked down using siRNA as follows. RAW264.7 cells were stably transfected with pSuppressor containing control siRNA, 4-1BB siRNA#1 or #2. 4-1BB mRNA was examined by RT-PCR. TNF production was measured in control and 4-1BB knockdown cells after LPS treatment for 24 hours. The siRNA very effectively knocked down 4-1BB in RAW264.7 cells, but the knockdown had no effect on LPS-induced TNF production (FIG. 5A-B). 4-1BB must then not be involved in LPS-induced TNF production in macrophages, and thus it has no link with 4-1BBL in the regulation of sustained TNF production. In sum, the 4-1BB-independent function of 4-1BBL in sustaining TNF production in LPS-treated macrophages was also observed in the RAW264.7 macrophage cell line by knocking down 4-1BBL and 4-1BB with siRNA (FIG. 4A-, C & FIG. 5A-B).

D. Transcriptional Studies

To determine whether 4-1BBL affects TNF expression at transcriptional and/or post-transcriptional stages, Tnf mRNA in 4-1BBL-deficient and wild-type macrophages was measured after various periods of LPS treatment (FIG. 3D). Expression of Tnf mRNA peaked at similar quantities in 4-1BBL-deficient and wild-type macrophages 2 hours after LPS stimulation; however, Tnf mRNA amounts in 4-1BBL-deficient cells were much lower than those in wild-type macrophages during later times after LPS stimulation (FIG. 3D). These data are consistent with the TNF protein data (FIG. 3B), and indicate that 4-1BBL influences the expression of Tnf mRNA transcripts.

To determine whether 4-1BBL influences the transcription of Tnf, nuclear run-on analysis was performed. LPS triggered Tnf transcription 1 hour after LPS stimulation in both wild-type and 4-1BBL-deficient macrophages; however Tnf transcription was reduced in 4-1BBL-deficient macrophages at later times following LPS treatment (FIG. 3E).

To determine whether the lower Tnf mRNA quantities found at late times following LPS stimulation in 4-1BBL-deficient macrophages were also resulted from a change in mRNA stability, we measured the half-life of Tnf mRNA in 4-1BBL-deficient and wild-type macrophages three hours after LPS stimulation. 4-1BBL deletion had a substantial influence on the stability of Tnf mRNA (FIG. 3F). Therefore, 4-1BBL influences both transcriptional and post-transcriptional regulation of TNF production.

Whether 4-1BBL deletion affects LPS-induced activation of NF-κB and other transcription factors was determined by electromobility shift assays (EMSA) as follows. 4-1BBL−/− and wild type macrophages were stimulated with LPS for 1, 2, 4, 8 and 12 hours and were then harvested for nuclear extract preparation. EMSA were performed using radiolabeled-probes. Results indicate that 4-1BBL deletion had no influence on LPS-induced NF-kB activation, an early response which peaked at two hours (FIG. 3G). This is consistent with the data in FIG. 3B, which shows that LPS-induced TNF production was not affected by 4-1BBL deletion in the first three hours. 4-1BBL deletion also had little or no effect on LPS-induced AP-1 activity (FIG. 3G). By contrast, LPS-induced CREB and C/EBP activation, which occurred after 8 hours of LPS stimulation, were significantly inhibited by 4-1BBL deletion (FIG. 3G). The impaired CREB and C/EBP activation may contribute to the defect in sustained TNF production in 4-1BBL-deficient macrophages.

To examine the LPS-induced signaling pathways, the NF-κB and MAP kinase pathways, peritoneal macrophages from wild type and 4-1BBL KO mice were treated with LPS (100 ng/mL) for 0.5, 1, 2, 4 and 6 hours, and cell lysates were immunoblotted with anti-IκB-α, antiphospho-ERK (p-ERK), anti-phospho-JNK (p-JNK), anti-phospho-p38 (p-p38), anti-4-1BBL, or anti-GAPDH antibodies. Results indicate that the rate of degradation of the inhibitor of NF-kB (IκB-α) was the same in 4-1BBL KO and wild type cells (FIG. 3H), while the level of ERK1/2, JNK1/2, and p38 MAP kinase activation (phosphorylation) in 4-1BBL KO cells was equal or slightly less than that in wild type cells (FIG. 3H).

Thus the early signal downstream of TLR4 appears not to be affected by 4-1BBL deletion, and the data obtained from analyzing the activation of signaling pathways (FIG. 3H) and transcription factors (FIG. 3G) are consistent with that obtained from analyzing TNF production (FIG. 3B). Further, it supports the notion that the early signaling of TLR4 is intact in 4-1BBL knockout macrophages.

### Example 5

### 4-1BBL Interacts with TLR2, TLR3, TLR4 and TLR9 and is Involved in TLR2-, TLR3-, TLR4- and TLR9-mediated TNF Production in Macrophages

To determine whether 4-1BBL is selectively involved in TLR4-mediated cellular responses, HA-4-1BBL was co-expressed with flag-TLR2, flag-TLR3, flag-TLR4, or flag-TLR9 as follows. 293T cells were transfected with the empty (control) or HA-4-1BBL expression vector, together with flag-TLR2, flag-TLR3, flag-TLR4, or flag-TLR9. The cells were lysed 24 hours after transfection. Cell lysates were immunoblotted with anti-flag antibodies, or were immunoprecipitated with anti-HA and then immunoblotted with anti-HA and anti-flag. Results indicate that all TLRs tested were pulled down by 4-1BBL in the co-immunoprecipitation assays (FIG. 6A). The co-immunoprecipitation has specificity because flag-IL-3R did not co-immunoprecipitate with 4-1BBL (FIG. 6A).

To determine whether 4-1BBL is involved in TLR2-, 3-, 4-, or 9-mediated cellular responses, the following experiment was conducted. Wild type and 4-1BBL−/− macrophages were treated with Pam3 (1 μg/mL), PolyI:C (25 μg/mL), LPS (100 ng/mL), R848 (100 nM), CpG (5 μg/mL), IL-1β (10 ng/mL), or untreated culture media (None). TNF production was measured after 24 hours of treatment. Results indicate that the Pam3 (TLR2 ligand)-, PolyI:C (TLR3 ligand)-, R848 (TLR7&8 ligand)-, CpG (TLR9 ligand)-induced production of TNF was reduced in 4-1BBL KO cells (FIG. 6B). The effect is TLR specific because IL-1β-induced TNF production was normal in 4-1BBL KO cells (FIG. 6B). Therefore, 4-1BBL is involved in the signaling of many, if not all, different TLRs.

### Example 6

### 4-1BBL Induction and Cell Surface Location is Required for Sustained TNF Production in LPS-treated Macrophages

4-1BBL is undetectable in most tissues, and only low levels are expressed in macrophages and dendritic cells (see Futagawa et al., Int. Immunol. 14:275-286 (2002)). To analyze the role of 4-1BBL in sustained TNF production in LPS-treated macrophage, macrophages isolated from wild type, TLR4−/−, MyD88−/−, and TRIF−/− mice were treated with LPS for 0.5, 1, 2, 4, and 8 hours. 4-1BBL protein was analyzed by immunoblotting using anti-4-1BBL antibodies. GAPDH was used as the loading control. Results indicate that LPS induces quick expression of 4-1BBL in macrophages with a peak at 4 hours (FIG. 7A, left 1-5 or 1-6 lanes of all panels). The timing of 4-1BBL induction correlates well with the sustained TNF production that was impaired in 4-1BBL knockout macrophages (FIG. 3B). The LPS-induced 4-1BBL expression is TLR4-, MyD88-, and TRIF-dependent, as it is impaired in TLR4−/−, MyD88−/−, and TRIF−/− macrophages (FIG. 7A). 4-1BBL expression was induced by all TLR ligands tested, but not by IL-1β (FIG. 8A-F). It appears that 4-1BBL is post-translationally modified after its synthesis (probably by glycosylation), since the shifted protein bands were observed on SDS-PAGE (FIG. 7A).

The promoter of the gene encoding 4-1BBL contains a number of NF-κβ-binding sites, and LPS-induced 4-1BBL mRNA was effectively inhibited by the NF-κB inhibitor sulfasalazine (FIG. 7B) or the proteasome inhibitor MG-132 (data not shown). The p38 inhibitor SB203580, the Jnk inhibitor SP600125, and the Mek inhibitor PD98059 also had some inhibitory effects on 4-1BBL expression (FIG. 7B). LPS-induced 4-1BBL mRNA (T₁₁₂=67 min) was more stable than 4-1BBL mRNA (T_{1/2}=33 min) expressed by an adenoviral vector in non-stimulated macrophages, indicating that LPS-induced alterations in mRNA stability were also involved in LPS-induced 4-1BBL expression (FIG. 7C).

4-1BBL expression in LPS-treated peritoneal macrophages from wild type mice was analyzed by flow cytometry using anti-4-1BBL antibodies. An isotype antibody was used as control. Permeabilization with 0.1% saponin was used to measure total 4-1BBL. Results indicate that most of the LPS-induced 4-1BBL is located on the cell surface (FIG. 7D).

To determine whether the newly synthesized 4-1BBL needs to be on the cell surface in order to regulate the expression of TNF, peritoneal macrophages were preincubated with or without brefeldin A (3 μg/mL) for 1 hour and were then treated with LPS for the indicated times. Immunostaining was performed using anti-4-1BBL antibodies. 4-1BBL and TNF mRNA were analyzed by semiquantitative PCR. GAPDH was used as control. Brefeldin A is a specific inhibitor that blocks protein translocation to the cell surface by inhibiting its translocation from the endoplasmic reticulum to the Golgi apparatus Klausner et al., J. Cell Biol. 116:1071-1080 (1992) (FIG. 7E). Blocking the translocation of newly synthesized 4-1BBL to the cell surface did not affect the LPS-induced increase in 4-1BBL mRNA (FIG. 7F). Brefeldin A treatment did not influence LPS-induced TNF mRNA after 1 hour, but it did reduce TNF mRNA at 2, 4, and 6 hours (FIG. 7F), suggesting that not only the induction but also the cell surface location of 4-1BBL is required for sustained TNF production.

### Example 7

### Expression and Cross-Linking of 4-1BBL Triggers TNF Production

Since the induction of 4-1BBL is required for sustained TNF production in LPS-treated macrophages, whether overexpression of 4-1BBL can trigger TNF production was examined. 4-1BBL was expressed in macrophages by adenovirus-mediated gene delivery as described in Bukczynski et al., Proc. Natl. Acad. Sci. U.S.A. 101:1291-1296 (2004). Briefly, macrophages were infected with different doses of adenoviruses encoding nothing (control) or 4-1BBL, and the expression of 4-1BBL was determined by immunoblotting with anti-4-1BBL antibodies. TNF levels in the media 24 hours after viral infection were measured. Results indicate that 4-1BBL expression induced TNF production in a dose-dependent manner (FIG. 9A). The TNF induction was 4-1BBL specific, since there was no detectable TNF in the medium of cells infected with the control virus. Induction of TNF by 4-1BBL-expression did not require 4-1BB, as TNF production was similar in 4-1BB-deficient and wild-type macrophages infected with the adenovirus encoding 4-1BBL (FIG. 9B).

To examine whether TNF production mediated by 4-1BBL overexpression is TLR4-, MyD88-, and TRIF-dependent, wild type, TLR4−/− and TRIF−/− macrophages were infected with the control or 4-1BBL expressing virus, and the 4-1BBL levels in the cells and TNF levels in the media were measured. Results indicate that induction of TNF production by 4-1BBL expression is partially impaired in macrophages isolated from TLR4−/− mice (FIG. 9C) suggesting that in addition to TLR4 other TLRs that can interact with 4-1BBL may also participate in 4-1BBL-mediated TNF production. Indeed, 4-1BBL interacted with TLR2 and other TLRs when they were co-expressed in 293T cells (FIG. 6A) and TNF production induced by 4-1BBL over-expression was reduced in TLR2-deficient macrophages (FIG. 9D). These results support the interpretation that multiple TLRs are involved in 4-1BBL-mediated TNF production. Despite the involvement of TLRs, TRIF deficiency had no effect on 4-1BBL-induced TNF production (FIG. 9E). Since macrophages isolated from MyD88 mice cannot be efficiently infected by adenovirus, the requirement of MyD88 in 4-1BBL-induced TNF production has to be addressed by another method (see below).

4-1BBL is a TNF superfamily member (see Watts, T. H., Annu. Rev. Immunol. 23:23-68 (2005)). It may be similar to other members in this superfamily in that trimer formation is required for its function (see Rabu et al., J. Biol. Chem. 280:41472-41481 (2005)). To test this possibility, wild type macrophages were treated with 4-1BB-Fc chimera (a disulfide-linked homodimer of 4-1BB), anti-Fc antibodies, or 4-1BB-Fc and anti-Fc together, and TNF levels in the medium were measured. More specifically, macrophages were treated with goat anti-human Fc is antibodies (anti-Fc, 1.5 μg/mL), mouse 4-1BB-human Ig-Fc domain fusion protein (4-1BB-Fc, 5 μg/mL), 4-1BB-Fc and anti-Fc together, or untreated culture media. TNF levels in the media were measured 24 hours later. Results indicate that 4-1BB-Fc can crosslink two 4-1BBL molecules, but it did not induce TNF production (FIG. 9F). Further crosslinking 4-1BB-Fc with anti-Fc antibodies did induce TNF production, while anti-Fc antibodies alone did not have any effect (FIG. 9F). It appears that crosslinking of more than two 4-1BBL molecules is needed to trigger TNF production. The requirement of 4-1BBL in the crosslinking induced TNF production was confirmed by using 4-1BBL KO cells (FIG. 9F).

To determine whether 4-1BBL signaling needs MyD88 and TRIF, MyD88−/− and TRIF−/− macrophages were treated with 4-1BB-Fc, anti-Fc antibodies, and both 4-1BB-Fc and anti-Fc antibodies, and TNF levels in the media were determined. Results indicated that the 4-1BBL crosslinking-mediated TNF production is MyD88- and TRIF-independent (FIG. 9F). In contrast, TNF production triggered by 4-1BBL crosslinking was reduced by a statistically significant margin in TLR2-deficient and TLR4-deficient macrophages, compared with that in wild-type macrophages (FIG. 9F).

One interpretation of these data is that expression and subsequent oligomerization of 4-1BBL on the cell surface triggers MyD88- and TRIF-independent TNF production. Because TLR4 and other TLRs interact with 4-1BBL (FIGS. 1A, 1B, 1D and 6A), and because TLRs form dimers (see Medzhitov et al., Nature 388:394-397 (1997)), it is possible that the partial dependence of TLR4 in the TNF production that is mediated by 4-1BBL overexpression (FIG. 9C) is due to 4-1BBL-TLR4 interactions, which contribute to the crosslinking of 4-1BBL. This is supported by the finding that 4-1BBL overexpression-mediated TNF production was partially inhibited by deletion of either TLR4 or TLR2 (FIG. 9C-F).

Since 4-1BB-Fc and anti-4-1BBL only bind two 4-1BBL molecules, and their binding with 4-1BBL should inhibit 4-1BBL oligomerization or prevent 4-1BBL from interacting with other molecules such as TLRs, they were used to test this hypothesis. Macrophages were stimulated with LPS in the presence of 4-1BB-Fc or anti-4-1BBL antibodies as follows. Macrophages were incubated with LPS, LPS with 4-1BB-Fc, LPS with 0, 2, or 5 μg/mL anti-4-1BBL antibodies, or with untreated culture media for 24 hours. Results indicate that both 4-1BB-Fc and anti-4-1BBL antibodies inhibited LPS-induced TNF production (FIG. 9G).

As 4-1BBL was induced by a number of TLR ligands (FIG. 8A-F), TNF production by 4-1BBL-deficient macrophages stimulated with TLR2 ligand (Pam3), TLR3 ligand (poly I:C), TLR4 ligand (LPS), TLR7,8 ligand (R848) and TLR9 ligand (CpG) was examined. A reduction of TNF production was observed (FIG. 6B). These data indicate the involvement of 4-1BBL in TNF production mediated by different TLRs. Since TLRs located in the endosome or endoplasmic reticulum (ER), such as TLR9, are unlikely to interact with 4-1BBL located in the plasma membrane, the signaling of TLR9-induced 4-1BBL may depend on 4-1BBL's interactions with other TLRs on the cell surface. Consistent with this is the detection of a small but statistically significant reduction in TLR9-induced TNF production in TLR4 deficient macrophages when a low dose of CpG (1 μg/mL) was used (FIG. 6C-D). However, mechanism(s) other than the interaction between 4-1BBL and cell surface TLRs could be involved in 4-1BBL signaling because the reduction was not significant by TLR4-deletion when the cells were stimulated with high doses of CpG.

### Example 8

### Two Sequential TLR4 Complexes

Since 4-1BBL induction depends on the TLR/MyD88/TRIF pathway, and 4-1BBL-mediated signaling is independent from MyD88 and TRIF, but linked to its interaction with TLR, whether there are sequential TLR4 signaling complexes in LPS-treated macrophages was examined as follows. Macrophages were treated with LPS for 0.5, 1, 2, 4, 8 and 12 hours. Total cell lysates were (1) immunoblotted with anti-4-1BBL, anti-TLR4, and anti-MyD88; (2) immunoprecipitated with anti-TLR4 antibodies and then immunoblotted with anti-TLR4, anti-4-1BBL, and anti-MyD88; (3) immunoprecipitated with anti-MyD88 antibodies and then immunoblotted with anti-MyD88, anti-TLR4, and anti-4-1BBL; or (4) immunoprecipitated with anti-4-1BBL and then immunoblotted with anti-4-1BBL, ant-TLR4, and anti-MyD88. An isotype antibody was used as negative control for all immunoprecipitations. The positive controls for the immunoblotting against 4-1BBL or MyD88 are shown in the indicated boxes. Results indicate that LPS induced 4-1BBL expression between the two and eight hour exposure times, but had no effect on the protein levels of TLR4 or MyD88 (FIG. 10A, Cell lysates). TLR4 was associated with MyD88 between the half hour and one hour LPS exposure times, but disassociated thereafter, while the TLR4-4-1BBL complex appeared between the two and 12 hour LPS exposure times (FIG. 10A, IP:TLR4). The TLR4-MyD88 complex was confirmed by immunoprecipitation of MyD88 (FIG. 10A, IP: MyD88). MyD88 was not able to pull down 4-1BBL, indicating that MyD88 and 4-1BBL are not in the same TLR4 complex. Immunoprecipitation of 4-1BBL pulled down TLR4 in the 2-12 hour-long LPS-treated cells, but was not able to pull down MyD88, confirming that MyD88 and 4-1BBL are not in the same complex (FIG. 10A, IP: 4-1BBL). Therefore, there are two sequential TLR4 complexes in LPS-treated macrophages, one is the MyD88 complex that is responsible for the initial cellular responses, and the second is the 4-1BBL-TLR4 complex that is involved in sustaining TNF production.

To confirm that MyD88 is not able to interact with 4-1BBL, AU1-tagged MyD88 was over-expressed with flag-4-1BBL or flag-TLR4 as follows. 293T cells were transfected with the MyD88-AU1 expression vector together with the flag-4-1BBL or flag-TLR4 expression vector. The cells were lysed 24 hours after transfection, and cell lysates were subjected to immunoprecipitation with anti-AU1 antibodies followed by immunoblotting with anti-flag and antiAU1 antibodies. Results indicate that Flag-TLR4, but not flag-4-1BBL, was pulled down by MyD88 (FIG. 10B).

To search for proteins that interact with 4-1BBL, the interaction of 4-1BBL with TRAF2 and TRAF6 was examined as follows. 293T cells were transfected with the HA-4-1BBL expression vector together with TRAF2-myc or TRAF6-myc expression vector. Cell lysates were immunoprecipitated with anti-HA antibodies and immunoblotted with anti-myc and anti-HA antibodies. Results indicate that 4-1BBL interacts with TRAF6, but not TRAF2 (FIG. 10C).

Because of the interaction between 4-1BBL and TRAF6, whether TRAF6 is required for 4-1BBL-mediated TNF production was examined. SiRNA was used to knockdown TRAF6 in RAW264.7 macrophages as follows. Macrophages were transfected with TRAF6-siRNA#1 or #2, or control siRNA (C), and TRAF6 protein levels were analyzed by immunoblotting against TRAF6. TNF production levels in the control siRNA-, TRAF6-siRNA#1- or TRAF6-siRNA#2-transfected cells were measured 24 hours after incubating with anti-Fc, 4-1BB-Fc, 4-1BB-Fc and anti-Fc together, LPS, Poly I:C (25 μg/mL), IL-1β (10 ng/mL), or untreated culture media. IL-6 levels were measured when the cells were treated with media, LPS, or TNF (10 ng/mL). Both siRNAs effectively reduced TRAF6 protein levels in RAW264.7 cells (FIG. 10D). TRAF6 knockdown and control cells were treated with 4-1BB-Fc, anti-Fc antibodies, or 4-1BB-Fc and anti-Fc antibodies together, and TNF levels in the medium were measured. Knocking down TRAF6 blocked 4-1BBL crosslinking-mediated TNF production (FIG. 10D). As expected, knockdown of TRAF6 impaired LPS, PolyI:C, and IL-1β induced cytokine production, but has no effect on TNF-induced IL-6 production (FIG. 10D).

To elucidate the downstream events triggered by 4-1BBL, transcription factor activation was examined. Expression of 4-1BBL activated CREB and C/EBP, but not NF-κB (FIG. 10E), which is consistent with the observation that 4-1BBL deletion had no effect on LPS-induced NF-κB activation, but did impair CREB and C/EBP activation (FIG. 3F). Similarly, no IκBα degradation was observed in cells overexpressing 4-1BBL (FIG. 10F). Expression of 4-1BBL triggered some phosphorylation of p38, Jnk, and Erk (FIG. 10F), and inhibition of p38, Jnk, and Erk (but not NF-κB) reduced 4-1BBL-mediated TNF production (FIG. 10G) suggesting that MAP kinase pathways are involved in 4-1BBL-mediated TNF production. The 4-1BBL signaling pathway appeared to affect Tnf mRNA, as 4-1BBL deletion resulted in a reduction of LPS-induced TNF mRNA transcription at later times (FIG. 3E) and 4-1BBL overexpression increased Tnf mRNA quantities (FIG. 10H). The half-life of Tnf mRNA in cells overexpressing 4-1BBL and in cells treated with LPS cells was compared; Tnf transcripts exhibited similar half lives (FIG. 10H). As LPS is known to stabilize Tnf mRNA and 4-1BBL deletion reduced the stability of LPS-induced Tnf mRNA (FIG. 3F), 4-1BBL appears to be a mediator of LPS-induced Tnf mRNA stabilization. Collectively, the data described here show that the initial activation of TNF expression and the sustained production of TNF in macrophages are regulated by early and late phase signaling pathways (FIG. 10I).

The expression of pro-inflammatory cytokines is tightly controlled by gene activating and inactivating mechanisms. The study described here shows that the initial activation of TNF expression and the sustained production of TNF are regulated by early and later phase signaling pathways (FIG. 10I). The well-known signal pathway, TLR4/MyD88/TRIF, is responsible for the initiation and early phase expression of inflammatory genes. 4-1BBL is one of the early induced proteins and is only induced in the early phase of cell activation. The newly synthesized 4-1BBL is translocated onto the cell surface to generate a new phase of signaling for sustained TNF production. The interaction between 4-1BBL and TLR appears to be involved in the generation of the second phase signaling, and the role of TLR in the sequential signaling complex is most likely to assist the oligomerization of 4-1BBL. The signaling mechanism of the 4-1BBL complex is different from that of the initial TLR4 signaling because it is independent from MyD88 and TRIF. However, both phases of the signaling require TRAF6.

The production of inflammatory cytokines is part of the inflammatory process, which is characterized by an initial and then sustained phase that normally ends when there is a resolution of inflammatory trigger (see Triantafilou and Triantafilou, Trends Immunol. 23:301-304 (2002)). Oftentimes a breakdown in the regulation of inflammation occurs at the ending of the sustained inflammatory responses, and the prolonged sustention of TNF production is often associated with the pathology of many inflammatory diseases (see Vassalli, P., Annu. Rev. Immunol. 10:411-452 (1992)). Therefore, the identification of 4-1BBL as a key regulator in sustained TNF production provides a new therapeutic target for development of new interventions for inflammatory diseases.

### Example 9

### 4-1BBL Plays a Role in IL-6 Induction

Il-6 is a cytokine involved in inflammation. Increased IL-6 expression is implicated in the pathology of several disease processes, including rheumatoid arthritis (RA), systemic-onset juvenile chronic arthritis (JCA), osteoporosis, and psoriasis. To show that 4-1BBL participates in IL-6 induction, the following experiments were conducted. First, 4-1BBL−/− and wild type mice were injected intraperitoneally with 0.5 mg of LPS. Then sera were collected at 0, 2 and 6 hours and IL-6 levels were measured. Results indicated that 4-1BBL−/− mice produce significantly less IL-6 in response to LPS than wild type mice (FIG. 11A). When peritoneal macrophages from 4-1BBL−/− and wild type mice were treated with LPS (100 ng/mL) and the levels of IL-6 in culture media were examined at 3, 9 and 18 hours, results confirmed that macrophages from wild type mice produced significantly more IL-6 than macrophages from 4-1BBL−/− mice (FIG. 11B). These results were compared with the IL-6 production levels 24 hours after wild type and 4-1BBL−/− macrophages were treated with Pam3 (1 μg/mL), PolyI:C (25 μg/mL), LPS (100 ng/mL), R848 (100 nM), CpG (5 μg/mL), IL-1β (10 ng/mL), TNF-α (10 ng/mL), or untreated culture media (None). Wild type macrophages treated with Pam3, PolyI:C, LPS, R848, and CpG produced significantly more IL-6 than 4-1BBL−/− macrophages (FIG. 11C) In contrast, the level of IL-6 production by wild type and 4-1BBL−/− macrophages were comparable.

When macrophages were incubated with LPS, LPS with 4-1BB-Fc, LPS with 0, 2, or 5 μg/mL anti-4-1BBL antibodies, or with untreated culture media for 24 hours, and then IL-6 levels in the media were measured, results showed that macrophages incubated with LPS and 4-1BB-Fc produced significantly less IL-6 than macrophages incubated with LPS alone (FIG. 11D). Macrophages incubated with LPS and increasing concentrations of anti-4-1BBL antibodies also exhibited decreasing levels of IL-6 production (FIG. 11D).

To confirm the role of 4-1BBL in IL-6 production RAW 264.7 cells were stably transfected with pSuppressor containing control siRNA, or 4-1BBL-siRNA#1 or #2. IL-6 production in 4-1BBL knockdown cells treated with LPS for 3, 9 and 24 hours was measured and compared with that of control cells. Results indicated that RAW264.7 cells in which 4-1BBL expression is knocked down produces significantly less IL-6 than RAW264.7 cells transfected with control siRNA (FIG. 11E). These results were consistent with that seen for control and 4-1BBL knockdown cells treated with peptidoglycan (PG, 10 μg/mL), PolyI:C (25 μg/mL), LPS (100 ng/mL), CpG (5 μg/mL), or untreated culture media (None) for 24 hours (FIG. 11F).

### DOCUMENTS CITED

Galanos, C., Freudenberg, M. A. & Reutter, W. Galactosamine-induced sensitization to the lethal effects of endotoxin. Proc. Natl. Acad. Sci. USA 76, 5939-5943 (1979).
Beutler, B. Inferences, questions and possibilities in Toll-like receptor signaling. Nature 430, 257-263 (2004).
Cohen, J. The immunopathogenesis of sepsis. Nature 420, 885-891 (2002). 4.
Janeway, C. A., Jr. & Medzhitov, R. Innate immune recognition. Annu. Rev. Immunol. 20, 197-216 (2002).
Akira, S. & Takeda, K. Toll-like receptor signaling. Nat. Rev. Immunol. 4, 499-511 (2004).
Goodwin, R. G. et al. Molecular cloning of a ligand for the inducible T cell gene 4-1BB: a member of an emerging family of cytokines with homology to tumor necrosis factor. Eur. J. Immunol. 23, 2631-2641 (1993).
Alderson, M. R. et al. Molecular and biological characterization of human 4-1BB and its ligand. Eur. J. Immunol. 24, 2219-2227 (1994).
Beutler, B. et al. Identity of tumour necrosis factor and the macrophage-secreted factor cachectin. Nature 316, 552-554 (1985).
Watts, T. H. TNF/TNFR family members in costimulation of T cell responses. Annu. Rev. Immunol. 23, 23-68 (2005).
Xu, Y. et al. Structural basis for signal transduction by the Toll/interleukin-1 receptor domains. Nature 408, 111-115 (2000).
Futagawa, T. et al. Expression and function of 4-1BB and 4-1BB ligand on murine dendritic cells. Int. Immunol. 14, 275-286 (2002).
Klausner, R. D., Donaldson, J. G. & Lippincott-Schwartz, J. Brefeldin A: insights into the control of membrane traffic and organelle structure. J. Cell Biol. 116, 1071-1080 (1992).
Bukczynski, J., Wen, T., Ellefsen, K., Gauldie, J. & Watts, T. H. Costimulatory ligand 4-1BBL (CD137L) as an efficient adjuvant for human antiviral cytotoxic T cell responses. Proc. Natl. Acad. Sci. U. S. A 101, 1291-1296 (2004).
Rabu, C. et al. Production of recombinant human trimeric CD137L (4-1BBL). Crosslinking is essential to its T cell co-stimulation activity. J. Biol. Chem. 280, 41472-41481 (2005).
Medzhitov, R., Preston-Hurlburt, P. & Janeway, C. A., Jr. A human homologue of the Drosophila Toll protein signals activation of adaptive immunity. Nature 388, 394-397 (1997).
Triantafilou, M. & Triantafilou, K. Lipopolysaccharide recognition: CD14, TLRs and the LPS-activation cluster. Trends Immunol. 23, 301-304 (2002).
Vassalli, P. The pathophysiology of tumor necrosis factors. Annu. Rev. Immunol. 10, 411-452 (1992).
DeBenedette, M. A. et al. Analysis of 4-1BB ligand (4-1BBL)-deficient mice and of mice lacking both 4-1BBL and CD28 reveals a role for 4-1BBL in skin allograft rejection and in the cytotoxic T cell response to influenza virus. J. Immunol. 163, 4833-4841 (1999).
Hoebe, K. et al. Identification of Lps2 as a key transducer of MyD88-independent TIR signaling. Nature 424, 743-748 (2003).
Kim, S. O., Ono, K., Tobias, P. S. & Han, J. Orphan nuclear receptor Nur77 is involved in caspase-independent macrophage cell death. J. Exp. Med. 197, 1441-1452 (2003).
Han, J., Jiang, Y., Li, Z., Kravchenko, V. V. & Ulevitch, R. T. Activation of the transcription factor MEF2C by the MAP kinase p38 in inflammation. Nature 386, 296-299 (1997).
Ge, B. et al. TAB1beta (transforming growth factor-beta-activated protein kinase 1-binding protein 1beta), a novel splicing variant of TAB1 that interacts with p38alpha but not TAK1. J. Biol. Chem. 278, 2286-2293 (2003).

### Other Embodiments

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent language be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A pharmaceutical composition comprising a 4-1BBL blocking agent and a pharmaceutically acceptable carrier, wherein the 4-1BBL blocking agent is selected from the group consisting of (a) an antagonistic antibody specific for 4-1BBL; (b) an oligonucleotide effective to reduce expression from a 4-1BBL nucleic acid in a cell; and (c) a soluble 4-1BB.

2. The pharmaceutical composition of claim 1, wherein the antagonistic antibody is a chimeric or humanized antibody specific for 4-1BBL.

3. The pharmaceutical composition of claim 1 or 2, wherein the 4-1BBL has the sequence set out in SEQ ID NO: 1 or 2.

4. The pharmaceutical composition of claim 1, wherein the oligonucleotide has a sequence selected from the group consisting of SEQ ID NO: 10-15, 22-38, and 45-56.

5. The pharmaceutical composition of claim 1, wherein the soluble 4-1BB has a sequence that corresponds to (a) amino acid 1 to amino acid 186 of the human 4-1BB polypeptide; (b) amino acid 23 to amino acid 186 of the human 4-1BB polypeptide; (c) amino acid 1 to amino acid 187 of the mouse 4-1BB polypeptide; or (d) amino acid 24 to amino acid 187 of the mouse 4-1BB polypeptide.

6. The pharmaceutical composition of claim 5, wherein the soluble 4-1BB has the sequence set out in SEQ ID NO: 7, 8, 20 or 21.

7. A pharmaceutical combination comprising a carrier, the 4-1BBL blocking agent of claim 1, and a second medicament that is an anti-inflammatory drug.

8. The pharmaceutical combination of claim 7, wherein the anti-inflammatory drug is an antibody specific for tumor necrosis factor.

9. The pharmaceutical combination of claim 7 or 8, wherein the antagonistic antibody is a chimeric or humanized antibody specific for 4-1BBL.

10. The pharmaceutical combination of claim 7 or 8, wherein the 4-1BBL has the sequence set out in SEQ ID NO: 1 or 2.

11. The pharmaceutical combination of claim 7 or 8, wherein the oligonucleotide has a sequence selected from the group consisting of SEQ ID NO: 10-15, 22-38, and 45-56.

12. The pharmaceutical combination of claim 7 or 8, wherein the soluble 4-1BB has a sequence that corresponds to (a) amino acid 1 to amino acid 186 of the human 4-1BB polypeptide; (b) amino acid 23 to amino acid 186 of the human 4-1BB polypeptide; (c) amino acid 1 to amino acid 187 of the mouse 4-1BB polypeptide; or (d) amino acid 24 to amino acid 187 of the mouse 4-1BB polypeptide.

13. The pharmaceutical combination of claim 12, wherein the soluble 4-1BB has a sequence selected from the group consisting of SEQ ID NO: 7-8 and 20-21.

14. A chimeric or humanized antibody specific for 4-1BBL.

15. The antibody of claim 14, wherein the antibody can bind to a 4-1BBL polypeptide comprising SEQ ID NO: 1 or 2.

16. A 4-1BBL blocking agent having a sequence selected from the group consisting of SEQ ID NO: 10-15, 22-38, and 45-56.

17. A soluble 4-1BB having a sequence that corresponds to (a) amino acid 1 to amino acid 186 of the human 4-1BB polypeptide; (b) amino acid 23 to amino acid 186 of the human 4-1BB polypeptide; (c) amino acid 1 to amino acid 187 of the mouse 4-1BB polypeptide; or (d) amino acid 24 to amino acid 187 of the mouse 4-1BB polypeptide.

18. The soluble 4-1BB of claim 17 that has the sequence of SEQ ID NO: 7, 8, 20 or 21.

19. A method for reducing production of an inflammatory cytokine in a mammal comprising administering a 4-1BBL blocking agent to the mammal, wherein the 4-1BBL blocking agent is selected from the group consisting of (a) an antagonistic antibody specific for 4-1BBL; (b) an oligonucleotide effective to reduce expression from a 4-1BBL nucleic acid in a cell; and (c) a soluble 4-1BB.

20. The method of claim 19, further comprising identifying a mammal suffering from or at risk for an inflammatory condition prior to administering the 4-1BBL blocking agent.

21. The method of claim 19 or 20, wherein the inflammatory cytokine is tumor necrosis factor or IL-6.

22. The method of claim 19 or 20, wherein the mammal is a human.

23. The method of claim 19 or 20, wherein the antagonistic antibody is a chimeric or humanized antibody specific for 4-1BBL.

24. The method of claim 19, 20, or 23 wherein the 4-1BBL has the sequence of SEQ ID NO: 1 or 2.

25. The method of claim 19 or 20, wherein the 4-1BBL blocking agent has a sequence selected from the group consisting of SEQ ID NO: 10-15, 22-38, and 45-56.

26. The method of claim 19 or 20, wherein the soluble 4-1BB has a sequence that corresponds to (a) amino acid 1 to amino acid 186 of the human 4-1BB polypeptide; (b) amino acid 23 to amino acid 186 of the human 4-1BB polypeptide; (c) amino acid 1 to amino acid 187 of the mouse 4-1BB polypeptide; or (d) amino acid 24 to amino acid 187 of the mouse 4-1BB polypeptide.

27. The method of claim 26, wherein the soluble 4-1BB has the sequence of SEQ ID NO: 7, 8, 20 or 21.

28-62. (canceled)
